(19) United States
(12) Patent Application Publication
(10) Pub. No.: US 2005/0043369 A1
(43) Pub. Date: Feb. 24, 2005

(54) Bactericidal antimicrobial methods and compositions for use in treating gram positive infections

(75) Inventors: Penelope N. Markham, Oak Park, IL (US); Debbie C. Mulhearn, Wheaton, IL (US); Ekaterina A. Klyachko, Chicago, IL (US); Alexander A. Neyfakh, Chicago, IL (US); David Crich, Chicago, IL (US); Mohamed-Rami Jaber, Romeoville, IL (US); Michael E. Johnson, Winnetka, IL (US)

Correspondence Address: POTTER ANDERSON & CORROON LLP;ATTN: KATHLEEN W. GEIGER, ESQ., P.O. BOX 951, WILMINGTON, DE, 19899-0951, US

(73) Assignees: Protez Pharmaceuticals Inc., Malvern, PA (US); The Board of Trustees of The University of Illinois, Chicago, IL (US)

(21) Appl. No.: 10/897,873

(22) Filed: Jul. 23, 2004

Related U.S. Application Data
(63) Continuation of application No. 09/816,761, filed on Mar. 23, 2001, now abandoned.
(60) Provisional application No. 60/191,879, filed on Mar. 23, 2000.

(51) Int. Cl. A61K 31/44; A61K 31/27; A61K 31/16; A61K 31/175; A61K 31/325

(52) U.S. Cl. 514/357; 514/482; 514/614; 514/590

(57) ABSTRACT

The present invention relates generally to the field of bacteriology. More particularly, the present invention provides methods and compositions for increasing the effectiveness of existing antibacterial agents and methods of overcoming bacterial resistance. Specifically, the invention provides methods of enhancing the action of an antibacterial agent by use of an antibiotic potentiator. Compositions of antibiotic potentiators including an acyl hydrazide, an oxy amide, and an 8-hydroxy quinoline also are disclosed.

### BACKGROUND OF THE INVENTION

The present application claims priority to co-pending U.S. Provisional Patent Application Ser. No. 60/191,879 filed Mar. 23, 2000. The entire text of the above-referenced disclosure is specifically incorporated by reference herein without disclaimer.

The government may own rights in the present invention pursuant to grant number 1R43 AI48092-01 from the National Institute for Health.

1. Field of the Invention

The present invention relates generally to the field of bacteriology. More particularly, the present invention provides methods and compositions for increasing the effectiveness of existing antibacterial agents and methods of overcoming bacterial resistance.

2. Description of Related Art

Gram positive organisms, particularly Staphylococci, Streptococci, and Enterococci, are increasingly seen as the major aetiological agents in infectious diseases. In the hospital setting, Staphylococcus aureus and Enterococcus faecalis account for more than 50% of isolates from blood stream infections (Cormican and Jones, 1996). In community-acquired infections, Streptococcus pneumoniae remains a leading cause of illness and death (Centers for Disease Control, 1996). The ongoing and rapid emergence and spread of antibacterial resistance in these organisms is thus a problem of critical proportions.

There is a general consensus among physicians that treatment of a bacterial infection with a bactericidal agent is preferable to treatment with a static agent. Unfortunately, a large number of classes of antibacterial agents including antibiotics such as macrolides, ketolides, tetracyclines, chloramphenicol, lincosamides and oxazolidinones, are all generally bacteriostatic at clinically achievable concentrations to most pathogens. The reason for this is that the vast majority of these agents are directed at the bacterial ribosome which, given its substantial differences to mammalian ribosomes, offers the ideal selective target for antibacterial chemotherapy. By binding to different sites on the bacterial ribosome, these agents arrest bacterial cell growth by inhibiting protein synthesis. Importantly many species of bacteria in this arrested state of growth can survive long periods of time in the presence of the antibiotic, and, after cessation of therapy, can regrow and cause a relapse in infection.

Since the immune system of a patient can eradicate bacterial cells whose growth is inhibited by antibiotics, these antibiotics remain effective for the treatment of a variety of infections. However, there are many situations in which bacteria are inaccessible to immune effector cells and a bactericidal antibiotic is recommended for treatment, including bacterial endocarditis, osteomyelitis, sepsis in the immunocompromised individual and other types of chronic infections (Peterson and Shanholtzer, 1992).

Bacterial endocarditis is associated with an extremely high rate of mortality which can range from 15-40% (Dyson et al., 1999). Bacterial vegetations in infectious endocarditis (IE) protect the invading organism from host defenses making it necessary to administer a bactericidal rather than a bacteriostatic antibiotic to obtain a cure (Koenig and Kaye, 1961). The most common causes of endocarditis are Gram positive cocci including Staphylococcus, Enterococcus and Streptococcus (Saccente and Cobbs, 1996) and recommended therapy includes the glycopeptides teicoplanin or vancomycin; β-lactams including oxacillin and methicillin; aminoglycosides; rifampin or quinolones. Additionally, combinations of agents which demonstrate bactericidal activity against the aetiological agent have been successfully used to obtain a cure (Moellering et al., 1971). However, the increasing resistance of the aetiological agents of IE to these antibiotics is drastically limiting treatment options and there is serious concern that resistance may develop to all available antibiotics (Cormican and Jones, 1996). Considering that the mortality rate for IE prior to the antibiotic era was 100%, this is indeed a daunting prospect.

Osteomyelitis is another situation where use of a bactericidal agent is recommended (Peterson and Shanholter, 1992). This condition is usually diagnosed when stationary growths of bacteria have established in the bone complicating therapy. When chronic, this disease is notoriously resistant to antibiotics. The ultimate goal of osteomyelitis treatment is to eradicate infection and prevent recurrence using antibiotic therapy which typically extends for a number of weeks (Karwowska et al., 1998). The most common cause of osteomyelitis is Staphylococcus, and, as is the case with endocarditis, the emerging resistance of this pathogen to a number of antibiotics is drastically limiting therapeutic options.

Individuals immunocompromised due to immunosuppression, chemotherapy or a disease state such as AIDs or diabetes are particularly prone to nosocomially acquired Gram positive infections such as S. aureus and E. faecalis, which, together, account for more than 35% of blood stream isolates in the U.S. (Pfaller et al., 1998). Due to the necessity to completely eradicate an infection in these individuals, bactericidal antibiotics are recommended for therapy. It is feared that effective treatment options for this increasing population of individuals will become progressively limited due to the rapid emergence and dissemination of antimicrobial resistance in nosocomial pathogens.

It is evident from the discussion above, that the treatment of various bacterial infections via bactericidal agents is preferable to current treatments with bacteriostatic agents. Further, due to the rapid emergence and spread of antibiotic resistance in Gram positive organisms, there is an urgent need for new bactericidal methods and compositions that increase the bactericidal effects of antibacterial agents.

### SUMMARY OF THE INVENTION

Although antibiotic agents are effective in treating a large number of Gram positive infections, their activity can be limited by the fact that they are typically bacteriostatic at clinically achievable concentrations. Thus, the development of bactericidal antimicrobial agents effective against Gram positive infections is highly desirable. The present invention provides antibiotic potentiators which are bactericidal in combination with a number of classes of antibacterial agents including antibiotics such as macrolides, ketolides, oxazolidinones, lincosamides, chloramphenicol and tetracyclines and antiseptics and disinfectants. The antibiotic potentiator, combined with existing bacteriostatic antibiotics, antiseptics or disinfectants, can provide a valuable therapeutic alternative, particularly when resistance to bactericidal antibiotics limits therapeutic options. The agents of the invention may also be used to treat fungal infections such as athlete's foot. As used herein, the term “antibiotic potentiator” includes any chemical composition that reduces the viability of bacterial cell growth when provided in combination with an antibiotic or antimicrobial agent. Thus, an antibiotic potentiator is meant to include, but is not limited to, chemical compositions that potentiate or increase the effect of a given antibiotic or antimicrobial agent in the treatment of bacterial infection, bacterial growth and the like.

In particular embodiments, an antibiotic potentiator is a chemical composition that increases the bactericidal effect of an antibacterial or antibiotic agent in a bacterial cell in comparison to the level of bactericidal activity of the antibiotic agent in the absence of the antibiotic potentiator. In one embodiment, a method for increasing the bactericidal action of an antibacterial agent comprises contacting a bacterium with an antibiotic potentiator, wherein the potentiator is an acyl hydrazide, an oxy amide, or an 8-hydroxy quinoline.

In a particular embodiment, the antibiotic potentiator is an acyl hydrazide of the general formula I: 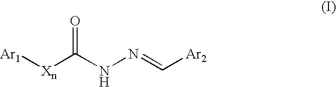
wherein Ar₁ and Ar₂ are independently aryl, substituted aryl, cycloalkyl, bicycloalkyl, substituted bicycloalkyls bicycloalkenyl, or substituted bicycloalkenyl, X is CH₂, C(CH₃)₂, NH, N-alkyl, N-phenyl, or S and n is 0 or 1. In a preferred embodiment, Ar₁ is selected from the group consisting of phenyl-, 4-toluoyl-, 4-isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo[2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo[2.2.1]heptane. In a preferred embodiment, Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl. In yet another preferred embodiment, the acyl hydrazide has the formula II. 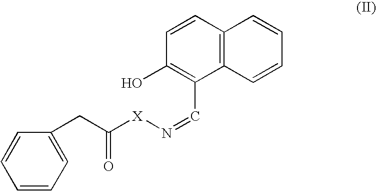
wherein X is CH₂, C(CH₃)₂, NH, N-alkyl or N-phenyl.

In another preferred embodiment, the acyl hydrazide has the formula (III) 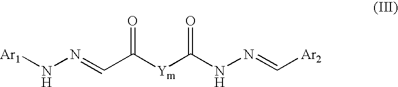
wherein Ar₁ and Ar₂ are independently aryl or substituted aryls, Y comprises one or more of C, N, and O and m is 1, 2, 3, 4, 5, 6, 7 or 8. It is preferred that Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo 2-phenol, 5-bromo-3-methoxy-2-phenol, 3-ethoxy-2-phenol-, 4,6-dimethoxy-2-phenol-, 4-methoxy-2-phenol and 2-thio-1-phenyl.

In other embodiments, the antibiotic potentiator is an oxy amide of formula IV: 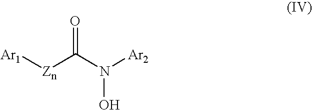
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1. It is preferred that Ar₁ is an anisole. n=0, and Ar₂ is a phenyl:

In other embodiments, the antibiotic potentiator is an 8-hydroxyquinoline of formula V: 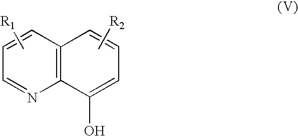
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group. A preferred compound is where R₁ is 2-(3,5-dimethyl-pyrazol-1-yl) and R₂ is H.

In particular embodiments, wherein a bacterium is contacted with an antibiotic potentiator, the bacterium is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter. In other embodiments, the antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

It is an aspect of this invention that the antibiotic potentiator be used without the addition of one or more antibacterial agents. It is contemplated that applications using the potentiator alone will require a high concentration of a transition metal such as iron, copper or manganese to have acceptable activity.

In another embodiment of the invention, a method of treating a subject with a bacterial infection comprises administering to the subject an antibacterial agent and an antibiotic potentiator, wherein the potentiator is an acyl hydrazide, an oxy amide, or an 8-hydroxy quinoline.

In a particular embodiment, the antibiotic potentiator is an acyl hydrazide of the general formula I: 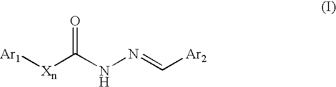
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1. In another preferred embodiment, the acyl hydrazide has the formula (m) 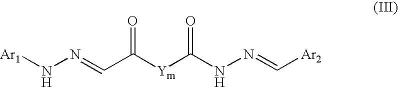
wherein Ar₁ and Ar₂ are independently aryl or substituted aryls, Y comprises one or more of C, N, and 0 and m is 1, 2, 3, 4, 5, 6, 7 or 8. In other embodiments, the antibiotic potentiator is an oxy amide of formula IV: 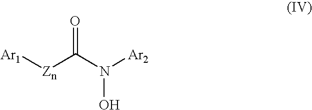
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1. In another embodiments, the antibiotic potentiator is an 8-hydroxyquinoline of formula V: 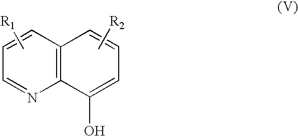
wherein R₁ and R₂ are independently H, alkyl, alkoxy, a halogen, substituted or unsubstituted I-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

In other embodiments, wherein treating a subject with a bacterial infection comprises administering to the subject an antibacterial agent and an antibiotic potentiator, the bacterial infection is of the genus Staphylococcus, Streptococcus, Enterococcus, Mycobacterium. Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter. In other embodiments, wherein treating a subject with a bacterial infection comprises administering to the subject an antibacterial agent and an antibiotic potentiator, the antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants. In further embodiments, treating a subject with a bacterial infection comprises administering to the subject a first and a second antibacterial agent and an antibiotic potentiator, wherein the first and the second antibacterial agents are selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein the first and the second antibacterial agents are chemically distinct compounds.

In yet another embodiment of the invention, a bactericidal pharmaceutical composition comprising an antibacterial agent and an antibiotic potentiator are provided, wherein the potentiator is an acyl hydrazide, an oxy amide, or an 8-hydroxy quinoline.

In a particular embodiment, the antibiotic potentiator is an acyl hydrazide of the general formula I: 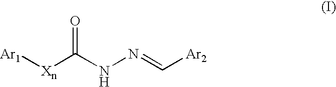
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1. In another preferred embodiment, the acyl hydrazide has the formula (III) 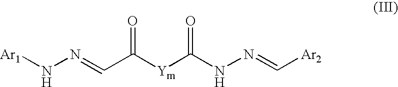
wherein Ar₁ and Ar₂ are independently aryl or substituted aryls, Y comprises one or more of C, N, and O and m is 1, 2, 3, 4, 5, 6, 7 or 8. In other embodiments, the antibiotic potentiator is an oxy amide of formula IV: 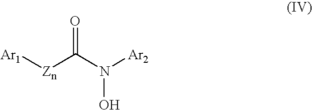
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1. In another embodiments, the antibiotic potentiator is an 8-hydroxyquinoline of formula V: 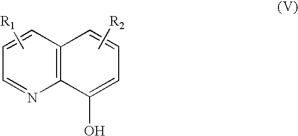
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

In other embodiments, wherein a bactericidal pharmaceutical composition comprising an antibacterial agent and an antibiotic potentiator is provided, the antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants. In further embodiments, a first and a second antibacterial agent are provided, selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein the first and the second antibacterial agents are chemically distinct compounds.

In another embodiment of the invention, provided is a method of screening for candidate acyl hydrazide antibiotic potentiators, oxy amide antibiotic potentiators or 8-hydroxy quinoline comprising contacting a bacterial cell with an antibacterial agent and an acyl hydrazide, an oxy amide, or an 8-hydroxy quinoline and comparing the bactericidal effect of the antibacterial agent in the presence and absence of the acyl hydrazide, oxy amide or 8-hydroxy quinoline, wherein a decrease in bacterial cell viability indicates the candidate acyl hydrazide, oxy amide or 8-hydroxy quinoline is an antibiotic potentiator. In particular embodiments, the antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants. In other embodiments, the bacterial cell is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

In yet another embodiment of the invention, a method of treating a subject for a bacterial biofilm infection is provided comprising administering to the subject an antibacterial agent and an antibiotic potentiator, wherein the potentiator is an acyl hydrazide, an oxy amide, or an 8-hydroxy quinoline.

In these embodiments, an acyl hydrazide has the general formula I: 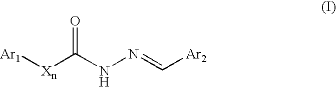
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1. In another preferred embodiment, the acyl hydrazide has the formula (III) 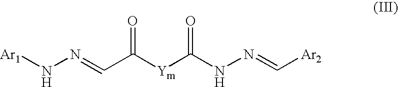
wherein Ar₁ and Ar₂ are independently aryl or substituted aryls, Y comprises one or more of C, N, and O and m is 1, 2, 3, 4, 5, 6, 7 or 8. In other embodiments, the oxy amide is of formula IV: 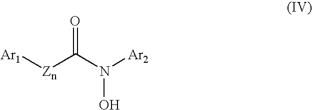
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1. In another embodiments, the 8-hydroxyquinoline is of formula V: 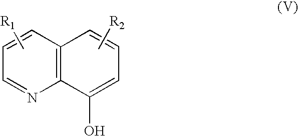
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

In particular embodiments, wherein a method of treating a subject for a bacterial biofilm infection is provided comprising administering to the subject an antibacterial agent and an antibiotic potentiator, the biofilm is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrohonomas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter. In specific embodiments, the biofilm infection is resistant to antibacterial agents. In certain embodiments, the infection is a chronic infection or persistent infection. In particular embodiments, the infection is endocarditis, osteomyelitis, an infection in a neutropenic subject or a biomaterial infection. In other embodiments, wherein a method of treating a subject for a bacterial biofilm infection is provided comprising administering to the subject an antibacterial agent and an antibiotic potentiator, the antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants. In yet other embodiments, provided are a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein the first and said second antibacterial agents are chemically distinct compounds.

In yet another aspect of the invention, provided is a pharmaceutical composition for inhibiting bacterial biofilm viability comprising an antibacterial agent and an antibiotic potentiator, wherein the potentiator is an acyl hydrazide, an oxy amide, or an 8-hydroxy quinoline.

In these embodiments, an acyl hydrazide has the general formula 1: 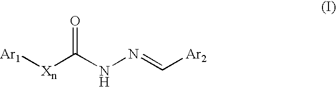
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1. In another preferred embodiment, the acyl hydrazide has the formula (III) 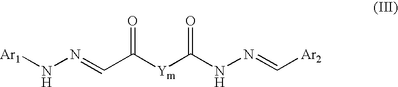
wherein Ar₁ and Ar₂ are independently aryl or substituted aryls, Y comprises one or more of C, N, and O and m is 1, 2, 3, 4, 5, 6, 7 or 8. In other embodiments, the oxy amide is of formula IV: 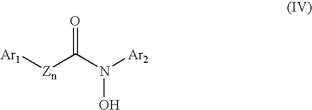
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1. In another embodiments, the 8-hydroxyquinoline is of formula V: 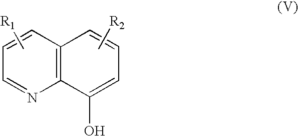
wherein R₁ and R₂ are independently H, allyl, alkoxy, a halogen, substituted or unsubstituted I-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

In particular embodiments, a biofilm is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter. In specific embodiments, an infection is resistant to antibacterial agents. In certain embodiments, an infection is a chronic infection or persistent infection. In certain other embodiments, an infection is endocarditis, osteomyelitis, an infection in a neutropenic subject or a biomaterial infection. In further embodiments, an antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants. In still further embodiments, provided are a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein the first and the second antibacterial agents are chemically distinct compounds.

In yet another aspect of the invention, provided is a method for increasing the bactericidal action of an antibacterial agent comprising: contacting a bacterial cell with an antibacterial agent; and contacting said bacterial cell with an acyl hydrazide potentiator, an oxy amide potentiator, or an 8-hydroxy quinoline potentiator, wherein said potentiator promotes the intracellular accumulation of a metal. It is preferred that the metal is a transition metal such as iron, copper, and/or manganese.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The general chemical structure of 14 of the 19 compounds screened. The library was screened for non-toxic compounds which, at a concentration of 10 μg/ml or less, reduced the viability of the S. aureus strain SA1199 (Kaatz et al., 1990) by three orders of magnitude in combination with a bacteriostatic concentration of erythromycin. Fourteen of the nineteen hits (74%), shared the structural moiety depicted in FIG. 1.

FIG. 2. Ortho-hydroxy aromatic functional group. Eleven of the fourteen compounds that shared the structural moiety depicted in FIG. 1., also shared an additional ortho-hydroxy aromatic group at position Ar₂ in FIG. 1.

FIG. 3. Lead compound INF 401. The activity of the hit compounds were compared by titration of the compounds in the presence of erythromycin at 4 μg/ml as described in Example 1. INF 401, shown in FIG. 3, was found to be the most active potentiator, active at concentrations as low as 0.15 μg/ml.

FIG. 4 Time kill study in S. aureus strain SA1199 of the effect of 5 μg/ml of INF 401 in combination with erythromycin at 2×MIC. One of these studies is presented.

FIG. 5A, FIG. 5B and FIG. 5C. Time kill studies of either FIG. 5A; chloramphenicol, FIG. 51; tetracycline or FIG. 5C; clindamycin alone, or in combination with 5 μg/ml of INF 401. Each antibiotic was at a concentration of 1×MIC.

FIG. 6. Time kill study of erythromycin (2×MIC), alone or in combination with 10 μg/ml of INF 402.

FIG. 7. Time kill study of the effect of NF 401 in combination with chloramphenicol. Exponentially growing cells (S. aureus strain 29213) were diluted to OD₆₀₀ 0.1 in LB supplemented with 20 μg/ml of chloramphenicol (Cm) in the presence or absence of INF 401 (2 μg/ml) and incubated with shaking at 37° C. At indicated time points cells were harvested, washed with LB and plated on LB agar plates. Number of colony forming units (CFUs) was determined after overnight incubation.

FIG. 8. Time kill study of the effect of iron on the bactericidal activity of INF 401. S. aureus strain 29213 was grown overnight in iron-limited Staphylococcal Siderophore Detection (SSD) medium (Heinrichs, 1999) supplemented with 2 μM FeCl₃, washed with SSD and diluted 1:100 into the fresh medium containing 0, 2 or 50 μm FeCl₃. Exponentially growing cells were diluted to OD₆₀₀ 0.01 in the corresponding medium supplemented when indicated with 20 μg/ml of chloramphenicol (Cm) and 2 μg/ml of INF 401. At 0, 1, 2 and 3.5 hours cells were plated in different dilutions to the LB agar plates and number of colony forming units (CFUs) was counted next day.

FIG. 9. Effect of INF 401 on the bactericidal activity of triclosan against S. aureus. Exponentially growing S. aureus were diluted to OD₆₀₀ 0.002 in LB supplemented with 50 μM FeCl₃ in the presence (INF 401) or absence (control) of 2 μg/ml of INF 401. Cells were incubated with shaking at 37° C. for 20 min in the presence of different concentrations of triclosan, washed twice with LB and plated on LB agar plates to count CFUs.

FIG. 10. Effect of INF 401 on bactericidal activity of hydrogen peroxide. Exponentially growing S. aureus were diluted to OD₆₀₀ 0.1 in LB supplemented with 50 μM FeCl₃ in the presence (INF 401) or absence (control) of 2 μg/ml of INF 401. Cells were incubated with shaking at 37° C. for 15 minutes in the presence of different concentrations of hydrogen peroxide (100 mM, 10 mM, 1 mM, 0.1 nM and 0.01 mM), washed twice with LB and plated on LB agar plates to count CFUs.

### DESCRIPTION OF ILLUSTRATIVE EMBODIMENTS

Due to the rapid emergence and spread of antibiotic resistance in Gram positive organisms, there is an urgent need for bactericidal antimicrobial agents effective against Gram positive infections. The present invention describes antibiotic potentiators which are bactericidal in combination with a number of classes of antibiotics including macrolides, ketolides, oxazolidinones, lincosamides, chloramphenicol and tetracyclines. The antibiotic potentiators are bactericidal in combination with antiseptics, disinfectants and other classes of antibacterial agents. Although these antibacterial agents are effective in treating a large number of infections their activity can be limited by the fact that they are typically bacteriostatic at clinically achievable concentrations and thus are ineffective in situations such as endocarditis, osteomyelitis and the treatment of immunocompromised individuals where it is imperative to eradicate an infection rather than simply stop bacterial growth. The antibiotic potentiator, combined with existing bacteriostatic antibiotics, can provide a valuable therapeutic alternative, particularly when resistance to bactericidal antibiotics limits therapeutic options. As used herein, the term “antibiotic potentiator” includes any chemical composition that reduces the viability of bacterial cell growth when provided in combination with an antibiotic or antimicrobial agent. Thus, an antibiotic potentiator is meant to include, but is not limited to, chemical compositions that potentiate or increase the effect of a given antibiotic or antimicrobial agent in the treatment of bacterial infection, bacterial growth and the like. In particular embodiments, an antibiotic potentiator is a chemical composition that increases the bactericidal effect of an antibiotic agent in a bacterial cell in comparison to the level of bactericidal activity of the antibiotic agent in the absence of the antibiotic potentiator.

The present inventors, by screening a chemical library for non-toxic compounds at a concentration of 10 μg/ml or less, identified 19 compounds which reduced the viability of S. aureus by three orders of magnitude in combination with a bacteriostatic concentration of erythromycin. Three of these hits did not share any obvious structural similarity with each other. Fourteen of the nineteen hits (74%) exhibited a striking similarity sharing the structural moiety of formula I:

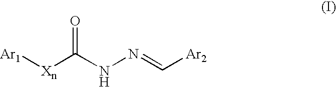

Eleven of these fourteen compounds share an ortho-hydroxy aromatic group at position R′. In certain compounds, X may comprises a CH₂, C(CH₃)₂, NH, N-alkyl, N-phenyl, O or S. Other compounds may comprise a -phenyl, -benzyl or -naphthyl at the R position. The lead compound, referred to herein as INF 401 (formula VI), was found to be the most active antibiotic potentiator, active at concentrations as low as 0.15 μg/ml.

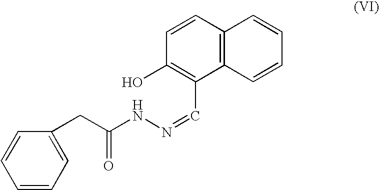

A second hit compound, referred to herein as INF 402 (formula VII), reduced the viability of S. aureus in combination with a bacteriostatic concentration of erythromycin INF 402, an oxy amide, is represented by the general formula IV but does not share structural similarity with the 14 compounds represented by the structural moiety of formula I.

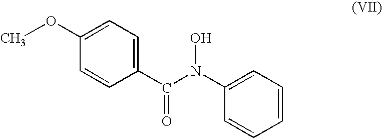

A third hit compound, referred to herein as INF 406 (formula VIII), reduced the viability of S. aureus in combination with a bacteriostatic concentration of erythromycin. INF 406, an 8-hydroxy quinoline, is represented by the general formula V but does not share structural similarity with the 14 compounds represented by the structural moiety of formula I.

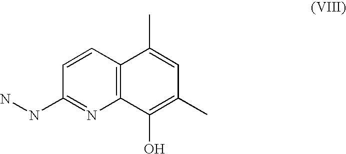

The present invention demonstrates that antibiotic potentiator compounds of the general chemical formula 1, formula III, and formula V, which, in combination with bacteriostatic concentrations of antibiotics, are synergistically and rapidly bactericidal to S. aureus. Data indicate also that the acyl hydrazide compounds of the present invention are effective also as antibiotic potentiators in S. pneumoniae.

It is contemplated further, that antibiotic potentiator compounds of formula I, formula M, and formula V are effective against a variety of Gram positive and Gram negative bacteria including Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella and Campylobacter in combination with various classes of antibacterial agents (e.g., macrolides, ketolides, tetracyclines, chloramphenicols, lincosamides, oxazolidinones, rifamycins, aminoglycosides, glycopeptides, daptomycins, fusidic acids, sulphonamides, cycloserines, lipopeptides, β-lactams, diaminopyrimidines, isonicotinic acids, nitrofurans, antiseptics and disinfectants).

Contemplated also in the present invention, is the inhibition or eradication of in vivo microbial biofilm growth. Microbial surface growth often takes the form of an organized biofilm (Stickler, 1999), in which the organisms are encased in a protective microenvironment. For example, bacteria that attach to surfaces, aggregate in a hydrated polymeric matrix of their own synthesis to form biofilms (Costerton et al., 1999), which provides an inherent resistance to antimicrobial agents. Such antimicrobial resistance results often in persistent or chronic bacterial infections. As described in Example 4 of the present invention, antibiotic potentiators in combination with antibacterial agents, are effective in eradicating S. aureus biofilms in vitro. Thus, in particular embodiments of the invention, an acyl hydrazide and/or oxy amide antibiotic potentiators in combination with antibiotic agents described herein are contemplated for treating or eradicating microbial biofilms in vivo.

A. INF 401 and its Analogs

As described in detail in Section F, Example 1, the inventors have identified nineteen non-toxic antibiotic potentiator compounds. Briefly, the inventors screened a chemical library for non-toxic compounds, which, at a concentration of 10 μg/ml or less, reduced the viability of the S. aureus by three orders of magnitude in combination with a bacteriostatic concentration of erythromycin. From the screening of this library, the inventors identified 19 non-toxic compounds which satisfied the criteria above. Three of these hits did not share any obvious structural similarity with each other. Fourteen of the nineteen hits (74%) exhibited a striking similarity sharing the structural moiety formula I. Eleven of these fourteen compounds shared an ortho-hydroxy aromatic group at position R′. Next the activity of the hit compounds were compared by titration of the compounds in the presence of erythromycin at 4 μg/ml as described above. The lead compound INF 401, formula II, was found to be the most active potentiator, active at concentrations as low as 0.15 μg/ml. INF 401 also potentiates the activity of bacteriostatic antibiotics in S. pneumoniae, indicating that a broad spectrum potentiator can be developed.

Identified also during screening of the chemical library were INF 402 and INF 406. The oxyamide INF 402 and the 8-hydroxyquinoline [NF 406 demonstrated the ability to also reduce the viability of S. aureus in the presence of bacteriostatic concentrations of erythromycin.

The inventors have thus identified a very promising class of acyl hydrazide compounds, which, in combination with a range of bacteriostatic antibiotics that target protein synthesis, is synergistically and rapidly bactericidal to S. aureus. It is contemplated, in the present invention, that acyl hydrazide antibiotic potentiators, oxyamide antibiotic potentiators and 8-hydroxy quinoline antibiotic potentiators in combination with bacteriostatic antibiotics will be developed that reduce the viability of a broad range of Gram positive bacteria. The following section provides details of strategies involving the development of lead compound INF 401 and its analogs as antibiotic potentiators.

1. Quantitative Structure Activity Analysis

The key structural features of the acyl hydrazide INF 401 lead to several obvious strategies for the probing of structure activity relationships and lead optimization. One of the more obvious of these concerns is the presumed cis/trans isomerization of the hydrazide, specifically around the hydrazone C═N double bond. NMR studies indicate that there is a mixture of isomers which are readily interconvertible and not separable at room temperature. This point is further reinforced by reported variable temperature NMR studies which revealed the barrier to interconversion to be below the threshold for the isolation of separate isomers at room temperature (Sandstrom, 1982). Since the cis/trans interconversion is more facile at 37° C., all of the INF 401 will be available for binding via equilibrium. Therefore, this leads to the conclusion that the use of a mixture of cis/trans acyl hydrazide isomers is of no consequence.

The development of the lead compound INF 401 and analogues have provided a lot of information regarding the structural characteristics necessary to have activity. Over 350 analogues of the lead acyl hydrazide INF 401, have been synthesized and tested, with approximately 140 of these hydrazides showing some degree of activity. With such a large number of compounds, a 3DQSAR/CoMFA study should generate worthy structural information. The overlay of pharmacaphore points is done with DISCO followed by 3D-QSAR/CoMFA generation, all accomplished within Sybyl 6.6 (available from Tripos, Inc., St. Louis, Mo.). Activity values (using the ERM4 concentration) of 80 hydrazides were used in the CoMFA study (leaving the other 60 compounds for a test set). This resulted in the statistical results being as follows: r²=0.706, F-value=43.302, and an SEE=0.285. If activity values at the ERM1 concentration were used (resulting in a smaller subset of the 80 hydrazones), the PLS analysis (statistical) shows the r²=0.998, the F-value=345.981, and the SEE=0.031, an incredible improvement on the statistical reliability. Despite the differences in the PLS analyses, the CoMFA results show nearly identical steric and electrostatic fields. These are summarized in Model 1 containing INF 401 as the basic acyl hydrazide. In addition to the CoMFA fields generated, CoMSIA fields (hydrogen donor/acceptor, hydrophobic, and electrostatic/steric) were also generated to further evaluate the structures. Unfortunately, only the hydrophobic properties were further mapped out, showing that the hydrophobic groups need to remain distant from the center acyl hydrazide moiety.

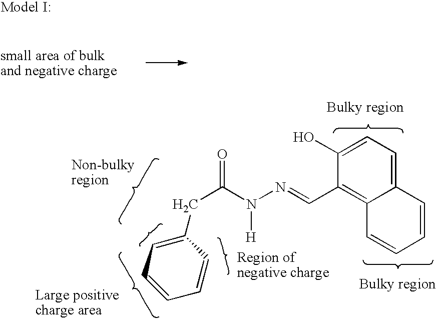

The above CoMFA model shows that there is a limitation in the size of the groups, and that the size of the aromatic groups on INF 401 is about as “bulky” as it likes it. Charge contributions are limited to the carbonyl side of the acyl hydrazide, with positive charge contributions weighing more than negative charge contributions. These results show that even with the large number of compounds used to generate these CoMFA fields, only small structural changes are suggested.

Further work and research on these acyl hydrazides identified one very important part of the mechanisms involved with the hydrazides: iron is necessary for the acyl hydrazide's activity. In fact, when the acyl hydrazide and iron are reacted, one product is found. Elemental analysis shows that the acyl hydrazide forms a specific complex with the iron. Therefore, it is probable that these acyl hydrazides chelate with transition metals such as iron, copper, and/or manganese, forming an acylhydrazide-metal complex. Complexes between acyihydrazides and metals such as these have previously been reported (Koh, Kon et al. 1998; Ranford, Vittal, and Wang 1998; Richardson and Bernhardt 1999; Lovejoy and Richardson 2000). Since activity of the acyl hydrazides relies upon the complexation with a transition metal such as iron, copper and/or manganese, further structural limitations can be suggested. One in being the obvious justification of the acyl hydrazide core, along with the ortho-hydroxyaromatic group on the hydrazine side, since these would be the atoms most likely to chelate with the iron or other transition metal. Activity testing of modified acyl hydrazides has also shown the acylhydrazide moiety should be intact, meaning that replacement of any of the atoms in the —C(═O)—N—N═C— chain with another atom such as oxygen, carbon, or a sulfonyl group, generally result in elimination of the activity. Therefore, the basic acyl hydrazide structure preferred for activity is (1), in Model 2 below. Model 2:

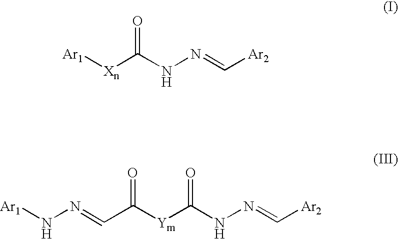

Starting with the acyl hydrazide building block, the X can either be a C or N, but not an 0, where n=0 or 1. There are also certain types of ring systems, Ar₁ and Ar₂, on either side of the hydrazone center that are more favored. Ar₁ can be: phenyl-, 4-toluoyl-4-isopropylphenyl-, 4-1-butylphenyl-, 2-anisole-, 4-ethylphenyl-, 3-chlorophenyl-, bicylco[2.2.1]heptane-, bicylco[2.2.1]hept-5-ene-, bicyclo[4.1.0]heptane-, hexahydro-2,5-methano-pentalene-, 1-pyridin-3-yl-,7,7-dimethyl-2-oxo-bicyclo[2.2.1]heptane, cyclohexane-, cycloheptane-, or 4,7,7-trimethyl-3-oxo-2-oxa-bicyclo[2.2.1]heptane-. Ar₂ is an ortho-hydroxy-aromatic group, which can include: 2-hydroxy-1-naphthyl-, 3,5-dichloro-2-phenol-, 4-diethylamino-2-phenol-, 3-methyl-2-phenol-, 4-methyl-2-phenol-, 5-methyl-2-phenol-, 5-bromo-2-phenol-, 5-bromo-3-methoxy-2-phenol-, 3-ethoxy-2-phenol-, 4,6-dimethoxy-2-phenol-, 4-methoxy-2-phenol-, 2-phenol-, or 2-thio-1-phenyl-. In addition to the acylhydrazones in Model 2 (1) listed, acyl dihydrazides, shown in Model 2 (III), are also found to have activity. The acyl dihydrazides may have any combination and number of Y's, where Y comprises one or more C, N, and/or 0 and where m=1, 2, 3, 4, 5, 6, 7, or 8. The Ar₁ and Ar₂ groups attached to either side of the acyl dihydrazide are the same as the Ar₂ ortho-hydroxy-aromatic groups listed for Model 2 (I).

There are two other classes of compounds found to be active, oxy amides (IV) and 8-hydroxyquinolines (V), shown in Model 3. As is with the acyl hydrazides, these two classes also rely upon transition metals such as iron, copper, or manganese for their activity. These two classes have not been synthetically explored yet for analogues. However, simple screening of readily available oxy amides and 8-hydroxyquinolines do suggest some structural limitations. The oxy amides are limited to the following base structure in (IV) possessing various substituents. From (IV), the oxy amides of particular interest are where Z comprises of one or more of C, N, and O, and n=0 or 1. The aromatic groups, Ar₁ and Ar₂, attached to either side of the oxy amide are independently phenyl, naphthyl, o-, -m-, or p-toluoyl, o-, m-, or p-anisole, any alkoxylphenyl, any halophenyl, a benzyl or a pyridinyl.

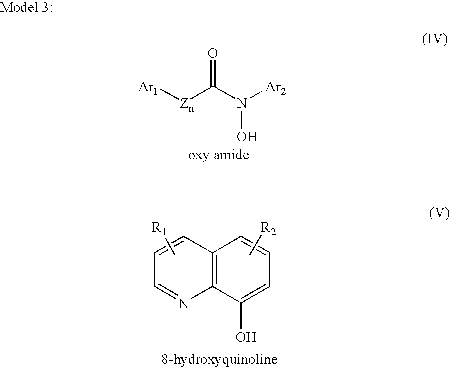

The 8-hydroxyquinolines, (V), are found to have greater solubility than the oxy amides and the acyl hydrazides, and are active with various substituents. One rather critical structural component is the 8-hydroxy-group. Methylation or further substitution at the 8-position eliminates the activity. This is not too surprising since any alteration at this position would greatly interfere with the compounds ability to chelate with a metal such as iron. Substituents that are likely to be favorable for the 8-hydroxyquinolines include: R₁ and R₂ independently being an H, alkyl, alkoxy, a halogen, a substituted or unsubstituted 1-allylphenyl, a benzyl, a hydrazino group (—NHNH2), a substituted hydrazino group, pyrazolyl, alkyl substituted pyrazolyl, an unsubstituted pyridazinyl group, or a substituted pyridazinyl groul. Further synthetic exploration and testing of the oxy amides and 8-hydroxyquinolines are needed for more structural qualifications.

B. Screening

In certain embodiments, the present invention concerns methods for identifying acyl hydrazide, oxy amide and 8-hydroxy quinoline antibiotic potentiators for use in reducing the viability of Gram positive bacteria. Examples of bacterial infections that may be treated by the inhibitors include but are not limited to those mediated by the genus of Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella and Campylobacter. The present invention thus provides methods of identifying acyl hydrazide antibiotic potentiators and oxy amide antibiotic potentiators. It is contemplated that this screening technique will prove useful in the general identification of any compound that will potentiate the bactericidal effects of antibacterial agents, wherein bacteria become less viable in the presence of a combination of an antibacterial agent and an antibiotic potentiator compound of the present invention.

Useful compounds in this regard will not be limited to those mentioned above. The active compounds may include fragments or parts of naturally-occurring compounds or may be only found as active combinations of known compounds which are otherwise inactive. However, prior to testing of such compounds in humans or animal models, it may be necessary to test a variety of candidates to determine which have potential.

Accordingly, in screening assays to identify pharmaceutical agents which potentiate the effects of antibacterial agents in bacteria, it is contemplated that compounds isolated from natural sources, such as animals, bacteria, fungi, plant sources, including leaves and bark, and marine samples may be assayed as candidates for the presence of potentially useful pharmaceutical agents.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

A particular library of compounds identified in the present invention are the acyl hyrdazides of the general formula I.

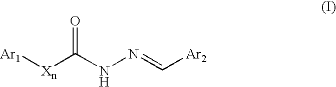

A second lead compound, not related structurally related to the acyl hyrdazides (formula I), is the oxy amide INF 402 represented by formula VII.

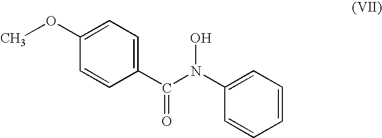

A third lead compound, not related structurally related to the acyl hyrdazides (formula I), is the 8-hydroxy quinoline INF 406 represented by formula VIII.

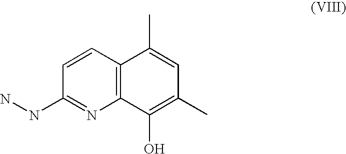

The screening of this library consisting of 9,600 compounds, has been completed. The chemical library was screened for non-toxic compounds at a concentration of 10 μg/ml or less. Positive hits were determined via a reduction in the viability of S. aureus by three orders of magnitude in combination with a bacteriostatic concentration of erythromycin. From the screening of this library, the inventors identified 19 non-toxic compounds which satisfied the criteria above.

In these embodiments, the present invention is directed to a method of screening for candidate acyl hydrazide antibiotic potentiators, oxy amide antibiotic potentiators or 8-hydroxy quinoline potentiators comprising:
(a) contacting a bacterial cell with an antibacterial agent;
(b) contacting a bacterial cell with said antibacterial agent and an acyl hydrazide, an oxy amide, or an 8-hydroxy quinoline; and
(c) comparing the bactericidal effect of said antibacterial agent in the presence and absence of said acyl hydrazide, oxy amide or 8-hydroxy quinoline.

A candidate acyl hydrazide, oxy amide or 8-hydroxy quinoline which decreases bacterial cell viability in the presence of an antibacterial agent, relative to bacterial cell viability in the presence of an antibacterial agent alone, indicates the candidate acyl hydrazide, oxy amide or 8-hydroxy quinoline is an antibiotic potentiator. Candidate acyl hydrazide, oxy amide and 8-hydroxy quinoline antibacterial potentiators that demonstrate bactericidal results, can further be tested in combination with various antibacterial agents and/or other candidate acyl hydrazide, oxy amide or 8-hydroxy quinoline compositions. It is understood in the present invention, that these screening assays may be performed using any desired antibacterial agent.

The candidate screening assay is quite simple to set up and perform. Thus, after obtaining a suitable test cell, one will admix a candidate acyl hydrazide, oxy amide or 8-hydroxy quinoline composition in the presence of an antibacterial agent with the cell, under conditions which would allow the uptake of the candidate composition and antibacterial agent. The ability of a candidate acyl hydrazide, oxy amide or 8-hydroxy quinoline composition to potentiate the effects of a given antibacterial agent can thus be measured by monitoring, for example viabilit

In an exemplary screening assay, in order to identify a candidate acyl hydrazide, oxy amide or 8-hydroxy quinoline composition as an antibacterial agent potentiator, the S. aureus strain SA1199 may be used. The procedure for screening is as follows: logarithmically growing S. aureus SA1199 are inoculated to a final OD₆₀₀ of 0.002 into 150 μl of LB medium containing a bacteriostatic concentration of an antimicrobial agent for that strain, for example, erythromycin (4 μg/ml) at four times the Minimal Inhibitory

Concentration (MIC). After overnight incubation the plates are shaken for 10 min to resuspend the cells and 2 μl was transferred to 150 μl of fresh LB medium (a 75-fold dilution of erythromycin concentration to 20-fold less than the MIC) and plates are examined for growth 20 hours later. In preliminary studies the inventors determined that under these conditions up to 2000 viable cells were transferred after incubation with erythromycin. As confirmed by broth microdilution, at least a 1000-fold reduction in viability was required to see no visible growth after 20 hours. Acyl hydrazide, oxy amide or 8-hydroxy quinoline antibiotic potentiators may be identified as those compounds that increase the bactericidal effect of the erythromycin.

“Effective amounts”, in certain circumstances, are those amounts effective at reproducibly increasing the bactericidal effect of the erythromycin in a bacterial cell in comparison to the level of bactericidal activity of the erythromycin in the absence of the candidate substance. Compounds that achieve significant changes in bactericidal activity of the erythromycin will be used. Thus, a battery of compounds may be screened in vitro to identify other agents for use in the present invention. The amounts of potentiators usefitl in this context may be determined by those of skill in the art and may vary from about 10 ng/ml to about 100 μg/ml. Thus it is contemplated that concentration ranges between these concentrations will be useful including but not limited to 20 ng/ml; 40 ng/ml; 60 ng/ml; 80 ng/ml; 100 ng/ml; 120 ng/ml; 140 ng/ml, 160 ng/ml; 180 ng/ml, 200 ng/ml, 350 ng/ml, 400 ng/ml, 450 ng/ml, 500 ng/ml, 550 ng/ml, 600 ng/ml, 650 ng/ml, 700 ng/ml, 750 ng/mL 800 ng/mL 900 ng/ml, 1 μg/ml, 5 μg/ml, 10 μg/ml, 15 μg/ml, 20 μg/ml, 25 g/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, and 100 μg/ml

A significant increase in bactericidal activity, e.g. as measured using growth curve analysis are represented by a reduction in bacterial growth of at least about 30%40%, and most preferably, by decreases of at least about 50%, with higher values of course being possible. Bacterial viability assays are well known in the art. Therefore, if a candidate substance exhibited potentiation of the bactericidal effects of an antibacterial agent in this type of study, it would likely be a suitable compound for use in the present invention.

Quantitative in vitro testing of the antibiotic potentiator is not a requirement of the invention as it is generally envisioned that the agents will often be selected on the basis of their known properties or by structural and/or functional comparison to those agents already demonstrated to be effective. Therefore, the effective amounts will often be those amounts contemplated to be safe for administration to animals in another context, for example, as disclosed herein. There is considerable information available on the use and doses of chemotherapeutic agents alone, which information may now be employed with the present invention.

C. Antibacterial and Antibiotic Classes

The present invention provides antibiotic potentiator compositions and methods which are bactericidal or exhibit increased bactericidal activity in combination with a number of classes of antibiotics and/or antiseptics or disinfectants. The following is a list of antibiotic classes contemplated to be effective in the present invention in combination with the acyl hydrazide, oxy amide and 8-hydroxy quinoline antibiotic potentiators described in section A. The antibiotic classes include but are not limited to those mediated by macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids and nitrofurans. Also contemplated to be effective in the present invention are antiseptics and disinfectants.

Macroides

Macrolides including erythromycin, azithromycin, clarithromycin, josamycin and oleandomycin have a broad spectrum of activity against most Gram positive pathogens, Neisseria, Haemophillus, Bordetella and Gram-positive and Gram-negative anaerobes. This class of antibiotics inhibits bacterial protein synthesis by binding to the SOS ribosomal subunit. Their principal use is in the treatment of infections due to Gram-positive cocci, notably of the skin, soft tissue and bone. They are also used to treat mycoplasma infections and act as substitutes for penicillin in allergic patients.

Tetracyclines

Tetracyclines including minocycline, doxycycline and tetracycline, exhibit a broad spectrum of predominantly bacteriostatic activity against both Gram-negative and Gram-positive bacteria. By binding to the 30S subunit of the bacterial ribosome, these antibiotics inhibit binding of aminoacyl tRNA and thus protein synthesis. Recommended uses include the treatment of common Gram positive infections, Chlamydia, Mycoplasma, Rickettsia and some Mycobacteria.

Chloramphenicol

Chloramphenicol exhibits bacteriostatic activity against a wide spectrum of Gram-positive and Gram-negative bacteria including Staphylococcus, Streptococcus, Enterococcus, Neisseria, Haemophillus, Escherichia, Klebsiella, and Pseudomonas. It is used for the treatment of typhoid fever, and severe Salmonella infections, meningitis and severe respiratory infections due to Haemophillus influenza.

Lincosamides

Lincosamides including clindamycin and lincomycin, inhibit bacterial protein synthesis by binding to the ribosome and inhibiting the peptidyl transferase reaction. They are active against Gram-positive bacteria and anaerobes. Their therapeutic indications are Staphylococcal infections, particularly osteomyelitis, penicillin-susceptible infections in allergic patients and anaerobic infections.

Oxazolidinones

Oxazolidinones including linezolid, which is currently under development, exhibit activity against a range of Gram-positive pathogens including Staphylococcus and Enterococcus. Inhibitors of bacterial protein synthesis, this class of antibiotics exhibits largely bacteriostatic effects.

Rifamycins

Rifamycins including rifampicin and rifabutin, specifically inhibit DNA-dependent RNA polymerase. They exhibit potent bactericidal activity against Gram-positive cocci and Mycobacteria. Due to the frequent selection of resistant mutants rifamycins are usually co-administered with a second antibiotic.

Aminoglycosides

Aminoglycosides including kanamycin, gentamycin, streptomycin, neomycin, tobramycin and spectinomycin exhibit potent bactericidal activity against a range of bacteria. They target the bacterial ribosome and inhibit protein synthesis. Depending on the particular antibiotic, aminoglycosides demonstrate activity against Staphylococcus and Mycobacteria with limited activity against other Gram positive bacteria. They are widely active against Enterobacteria and aerobic Gram-negative bacilli but exhibit no activity against anaerobes. Therapeutic indications include severe sepsis due to Enterobacteria or other aerobic Gram-negative infections. Additionally, some aminoglycosides are recommended for endocarditis, skin, and respiratory infections and tuberculosis.

Glycopeptides

Glycopeptides including vancomycin, teicoplanin and LY-3338 (in development), which inhibit the synthesis of peptidoglycan and assembly of the cell wall, exhibit a narrow spectrum of bactericidal activity against Gram-positive bacteria. They are administered for severe Staphylococcal and Enterococcal infections including endocarditis and catheter-related infections, as well as being administered prophylactically for certain surgical procedures and decontamination of bowels in neutropenic patients.

Daptomycin

The cyclic lipopeptide daptomycin, currently under development, exhibits a broad spectrum of bactericidal activity against aerobic and anaerobic Gram-positive pathogens including Staphylococcus, Enterococcus and Clostridium. This antibiotic inhibits an early stage of peptidoglycan synthesis and cell wall assembly.

Fusidanes

Fusidanes including helvolic and fusidic acid are active against Gram-positive bacteria and Gram-negative cocci. This antibiotic binds the elongation factor EF-G required for peptide translocation, thus inhibiting protein synthesis. Therapeutic indications include Staphylococcal osteomyelitis.

Sulphonamides

Sulphonamides including sulphathiazole and sulphamethoxazole, act as inhibitors of folic acid synthesis. They exhibit a broad spectrum of antibacterial activity including Streptococci and Staphylococci. Indications for use are restricted by the emergence of resistance. Their principal use is in the treatment of urinary tract infections, either alone or in combination with trimethoprim.

Cycloserine

Cycloserine, which inhibits peptidoglycan synthesis, is active against a wide range of Gram-negative and Gram-positive pathogens including Staphylococcus, Streptococcus and Enterococcus. It is used mainly for the treatment of tuberculosis in combination with other antibiotics.

β-lactams

β-lactams comprise a large group of agents including Penams, Penems, Carbapenems, carbapenams, Cephems, Clavams and Azetidinones. They inhibit the synthesis of cell wall peptidoglycan and are bactericidal. Due to their broad spectrum of antimicrobial activity they are widely used, especially for the treatment of Streptococcal infections, gonorrhea, Staphylococcal infections, anaerobic infections, Haemophilus influenza infections, and urinary tract infections.

Diaminopyrimidines

Diaminpyrimidines, including trimethoprim and cotrimethoxazole, inhibit folic acid synthesis. These agents exhibit activity against aerobic Gram-positive bacilli and cocci, including Staphylococcus. Indications for usage include enteric fever, the control of infections in neutropenic individuals and the topical treatment of burns.

Isonicotinic acid

Isonicotinic acid or Isoniazid, inhibits the synthesis of mycolic acid, a constituent of the cell wall of Mycobacterium tuberculosis. It is used as a first line defense against M. tuberculosis in combination with other antibiotics.

Nitrofurans

Nitrofurans including nitrofurazone and nitrofurantoin exhibit a broad spectrum of antimicrobial activity including Staphylococci and Enterococci, Enterobacteria and Clostridia. They are used for the treatment of urinary tract infections, intestinal infections and infections of the skin, eye and vagina.

Antiseptics and Disinfectants

Antiseptics and disinfectants comprises antibacterial agents which are contemplated to be effective in the present invention in combination with the acyl hydrazide, oxy amide and 8-hydroxy quinoline antibiotic potentiators. These compounds include, but are not limited to an alcohol, aldehyde, anilide, biguanide such as chlorhexidine, diamidine, halogen-releasing agent, silver compound, peroxygen compound such as hydrogen peroxide, phenol, bis-phenol such as triclosan, halophenol or quaternary ammonium compounds.

Chlorhexidine is one of the most widely used biocides in antiseptic products including handwashing and oral products and also as a disinfectant and preservative. It has a broad spectrum of antibacterial and antifungal activity.

Triclosan is widely employed in antiseptic soaps and handrinses. Generally considered to have a broad spectrum of activity, it is particularly active against Gram positive bacteria and exhibits little activity against some Gram negative pathogens and molds.

D. Combination Therapy

Bacterial intrinsic and acquired resistance to antibiotics represents a major problem in the clinical management of bacterial infections. A large number of classes of antibiotics, including macrolides, ketolides, tetracyclines, chloramphenicol, lincosamides and oxazolidinones, are all generally bacteriostatic at clinically achievable concentrations to most pathogens. Importantly many species of bacteria in this arrested state of growth (ie., bacteriostatic) can survive long periods of time in the presence of the antibiotic, and, after cessation of therapy, can regrow and cause a relapse in infection. It is therefore clinically desirable to treat various bacterial infections with bactericidal agents rather than current treatments using bacteriostatic agents. Thus, one of the goals of the present invention is improving the efficacy of existing bacteriostatic compounds against bacterial infection.

One way of achieving such a beneficial therapeutic outcome is to combine traditional antibiotics with agents that potentiate or increase the effects of bactericidal agents. Such combination antibiotic therapy would be conceptually similar to the already widely used combinations of β-lactam or cephalosporin antibiotics with inhibitors of β-lactamase. In fact, one such combination, augmentin, has become one of the most frequently prescribed antibiotic preparations in the United States. More particularly, it is a goal of the present invention to improve the efficacy of antibiotic agents. The inventors contemplate that the clinical use of antibiotics in combination with an antibiotic potentiator should dramatically improve the clinical efficacy of these antibiotics by both reducing their effective concentration several fold (shifting it well below their practically achievable tissue levels) and preventing the emergence of drug-resistant variants. More specifically the present invention provides combinations of antibiotics and antibiotic potentiators for combating Gram positive infection.

To kill bacterial cells, inhibit bacterial cell growth, or otherwise reverse or reduce the suppressing effect on the emergence of drug-resistant variants bacterial species using the methods and compositions of the present invention, one would generally contact a “target” cell with an antibiotic potentiator and at least one antibiotic. The compositions would be provided in a combined amount effective to kill or inhibit bacterial cell growth. This process may involve contacting the cells with the antibiotic potentiator, the antibiotic(s) or other bactericidal factor(s) and/or transition metal(s) at the same time. This may be achieved by contacting the cell with a single composition or pharmacological formulation that includes both agents, or by contacting the cell with two distinct compositions or formulations, at the same time, wherein one composition includes the antibiotic potentiator and the other includes the antibiotic.

The antibiotic potentiator treatment may precede or follow the other antibiotic, antibacterial agent, or transtion metal by intervals ranging from minutes to hours to days. In embodiments where the antibiotic and antibiotic potentiator are administered separately, one would generally ensure that a significant period of time did not expire between the time of each delivery, such that the antibiotic and antibiotic potentiator would still be able to exert an advantageously combined effect on abrogating the bacterial infection. In such instances, it is contemplated that one would administer both modalities within about 12-24 hours of each other and, more preferably, within about 6-12 hours of each other, with a delay time of only about 12 hours being most preferred. It may be that in order to sensitize the bacterial cells to the antibiotic treatment, the antibiotic potentiator is administered for a sufficient period of time (1, 2 3, 4, 5, 6, 7, 8, 12, 24 hours) prior to the antibiotic treatment. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Equally it may be necessary to administer multiple doses of the antibiotic potentiator in order to sensitize the bacterial cells to the antibiotic treatment.

It also is conceivable that more than one administration of either antibiotic potentiator or the antibiotic will be desired. Various combinations may be employed, where the antibiotic potentiator is “A” and the antibiotic is “B”, as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve bacterial cell killing, both agents are delivered to a cell in a combined amount effective to kill the cell and remove the infection.

Agents or factors suitable for use in a combined therapy are any antibiotic chemical compound or treatment method that induces damage when applied to a bacterial cell. More particularly, the present invention uses antibiotics in combination with the antibiotic potentiator of the present invention. Such antibiotics include but are not limited to the following antibiotic classes: macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans and also antiseptics, disinfectants and the like.

In treating a bacterial infection according to the invention, one would contact the bacterial cells with an antibiotic agent in addition to the antibiotic potentiator. This may be achieved by contacting the bacterial cells with the agent by administering to the subject a therapeutically effective amount of a pharmaceutical composition comprising an antibiotic compound and a therapeutically effective amount of the antibiotic potentiator.

The skilled artisan is directed to “the Physicians Desk Reference” 52nd Edition, in order to find detailed specific disclosure regarding particular antibiotics. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA Office of Biologics standards.

The inventors contemplate that the regional delivery of antibiotic potentiator and/or the antibiotic compositions to patients with Gram positive bacterial infection will be a very efficient method for delivering a therapeutically effective composition to counteract the clinical disease. Alternatively, systemic delivery of antibiotic potentiator and/or the antibiotic may be the most appropriate method of achieving therapeutic benefit from the compositions of the present invention.

E. Pharmaceutical Administration

Pharmaceutical compositions of the present invention will generally comprise an effective amount of the antibiotic potentiator dissolved or dispersed in a pharmaceutically acceptable carrier or aqueous medium. The pharmaceutical composition may further comprise an antibiotic composition or antibacterial composition.

Transition metals such as iron, copper and manganese are known to catalyze free radical formation (e.g. the Fenton reaction) (Olanow et al., 1994). It is contemplated that a transition metal or transition metal complex be part of the pharmaceutical composition of the current invention. Solutions containing FeCl₃ or CuSO₄ or any metal that forms free metal ions are preferred.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifingal agents, isotonic and absorption delaying agents and the like. The use of such media and agents for pharmaceutical active substances is well known in the art Except insofar as any conventional media or agent is incompatible with the active ingredient, its use in the therapeutic compositions is contemplated. Supplementary active ingredients also can be incorporated into the compositions.

The antibiotic potentiator of the present invention will often be formulated for parenteral administration, e.g., formulated for injection via the intravenous, intramuscular, sub-cutaneous or other such routes, including direct instillation into an infected or diseased site. The preparation of an aqueous composition that contains an antibiotic potentiator as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection also can be prepared; and the preparations also can be emulsified Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The antibiotic potentiator compositions can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques which yield a powder of the active ingredient plus any additional desired ingredient from a previously sterile-filtered solution thereof.

It is an aspect of this invention that the antibiotic potentiator, be applied in a non-systemic application. These include, but are not limited to topical applications, anti-acne, handwashing, eye washing, at a surgical site, surface infection, tissue trauma or other wound.

Nasal and aerosol and administrations are also contemplated. The non-systemic application may be a cream, lotion, paste, ointment, spray, powder, solution, colloidal suspension and the like. Suitable pharmaceutical vehicles may be used to prepare the antibiotic potentiator formulations of the invention, including petrolatum, whitepsol ointment, various lotions, emulsion bases, creams, and the like.

Lotions include those suitable for application to the skin or eye. An eye lotion may comprise a sterile aqueous solution. Lotions or liniments for application to the skin optionally include an agent to hasten drying and to cool the skin, such as an alcohol or acetone, and/or a moisturizer such as glycerol or an oil such as castor oil or arachis oil. Creams, ointments or pastes are semi-solid formulations of the active ingredient for external application. They may be made by mixing the antibiotic potentiator, antibacterial agent and optionally a metal cation in finely-divided or powdered form, alone or in solution or suspension in an aqueous or non-aqueous fluid. Hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel may also be included in the cream, ointment or paste. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Upon formulation, solutions will be administered in a manner compatible with the dosage formulation and in such amount as is therapeutically effective. Formulations are easily administered in a variety of dosage forms, such as the type of injectable solutions described above, but drug release capsules and the like also can be employed.

Suitable pharmaceutical compositions in accordance with the invention will generally include an amount of the antibiotic potentiator admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. It should be appreciated that, for human administration, preparations should meet sterility, pyrogenicity, general safety and purity standards as required by the FDA Office of Biological Standards.

The therapeutically effective doses are readily determinable using an animal model, as shown in the studies detailed herein. Experimental animals bearing bacterial or fungal infection are frequently used to optimize appropriate therapeutic doses prior to translating to a clinical environment. Such models are known to be very reliable in predicting effective anti-bacterial and anti-fungal strategies.

In addition to the compounds formulated for parenteral administration, such as intravenous or intramuscular injection, other pharmaceutically acceptable forms also are contemplated, e.g., tablets or other solids for oral administration, time release capsules, liposomal forms and the like. Other pharmaceutical formulations may also be used, dependent on the condition to be treated.

For oral administration the antibiotic potentiator of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The present invention also provides therapeutic kits comprising the antibiotic potentiator described herein. Such kits will generally contain, in suitable container means, a pharmaceutically acceptable formulation of at least one antibiotic potentiator in accordance with the invention. The kits may also contain other pharmaceutically acceptable formulations, such as those containing antibiotics and any one or more of a range of chemotherapeutic drugs.

The kits may have a single container means that contains the antibiotic potentiator, with or without any additional components, or they may have distinct container means for each desired agent. Certain preferred kits of the present invention include a antibiotic potentiator, packaged in a kit for use in combination with the co-administration of an antibiotic. In such kits, the antibiotic potentiator and the antibiotic may be pre-complexed, either in a molar equivalent combination, or with one component in excess of the other; or each of the antibiotic potentiator and antibiotic components of the kit may be maintained separately within distinct containers prior to administration to a patient. Other preferred kits include any antibiotic potentiator of the present invention in combination with a “classic” chemotherapeutic agent. This is exemplary of the considerations that are applicable to the preparation of all such antibiotic potentiator kits and kit combinations in general.

When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means.

The container means of the kit will generally include at least one vial, test tube, flask, bottle, syringe or other container means, into which the antibiotic potentiator, and any other desired agent, may be placed and, preferably, suitably aliquoted. Where additional components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the antibiotic potentiator to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Screening of the Chemical Library

The inventors screened a chemical library for compounds that, in combination with a bacteriostatic concentration of a translational inhibitor, are bactericidal to S. aureus. The library screened (Diverse ™96) is ideal for identifying novel drug leads, being constructed to maximize structural diversity of compounds of molecular masses of 200-700 while excluding clearly toxic and unstable molecules. The inventors have previously used this library successfully to identify novel inhibitors of the NorA multidrug transporter in S. aureus (Markham and Neyfakh, 1996).

The library was screened for non-toxic compounds, which, at a concentration of 10 μg/ml or less, reduced the viability of the S. aureus strain SA1199 (Kaatz et at., 1990) by three orders of magnitude in combination with a bacteriostatic concentration of erythrornycin. The macrolide erythromycin was chosen in the initial screen for two reasons: first, its mechanism of inhibition of protein synthesis is well characterized and second, as a macrolide, erythromycin is representative of an expanding class of agents which includes the second most prescribed antibiotic, azithromycin.

The procedure for screening was as follows: logarithmically growing S. aureus SA1199 were inoculated to a final OD₆₀₀ of 0.002 into 150 ll of LB medium containing erythromycin (4 μg/ml) at four times the Minimal Inhibitory Concentration (MIC). After overnight incubation the plates were shaken (Brinkmann Titermix 100) for 10 min to resuspend the cells and 2 μl was transferred to 150 μl of fresh LB medium (a 75 fold dilution of erythromycin concentration to 20 fold less than the MIC) and plates examined for growth 20 h later. In preliminary studies the inventors determined that under these conditions up to 2000 viable cells were transferred after incubation with erythromycin. As confirmed by broth microdilution, at least a 1000-fold reduction in viability was required to see no visible growth after 20 h.

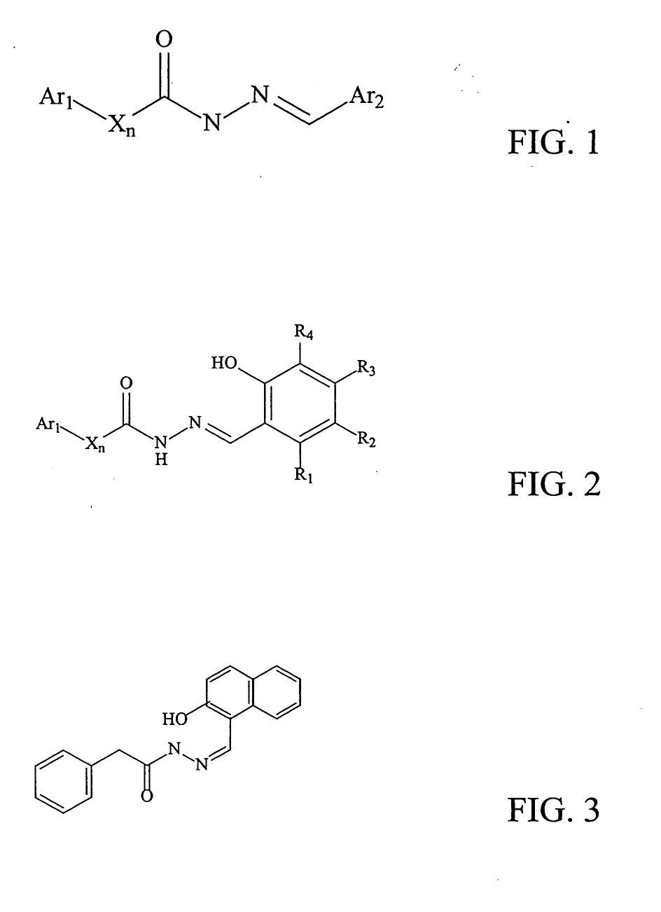

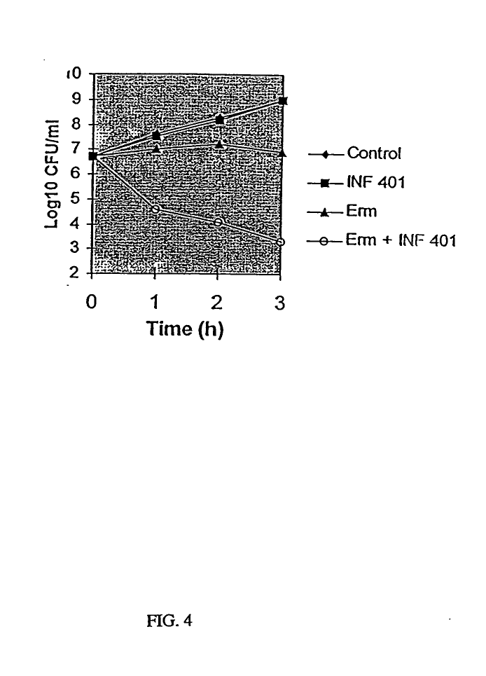

From the screening of this library, the inventors identified 19 non-toxic compounds which satisfied the criteria. Three of these hits did not share any obvious structural similarity with each other. Remarkably, fourteen of the nineteen hits (74%) exhibited a striking similarity sharing the structural moiety shown in FIG. 1. Eleven of these fourteen compounds shared the additional phenol group with an o-hydroxy group as shown in FIG. 2.

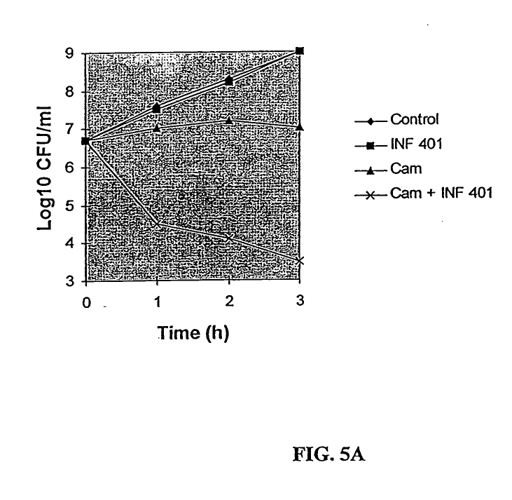

Next the activity of the hit compounds was compared by titration of the compounds in the presence of erythromycin at 4 μg/ml as described above. INF 401, shown in FIG. 3, was found to be the most active potentiator, active at concentrations as low as 0.15 μg/ml.

The next most active class of hits was the 8-hydroxquinoline, IF 406 which was active to 0.6 μg/ml. INF 402, the oxy amide was active to 1.25-2.5 μg/ml.

### Example 2

### Rapid Bactericidal Activity of INF 401 in Combination with Erythromycin

The inventors selected the most active compound, INF 401, representative of the largest class of active hits, for further characterization. Checkerboard titration was performed with INF 401 and erythromycin to quantitate the effects of the combination on Staphylococcal growth using two-fold serial broth microdilution. After overnight incubation, cells were transferred to fresh medium, as described above, to determine those combinations that were bactericidal to S. aureus. Erythromycin alone resulted in a greater than 1000-fold decrease in cell viability at concentrations of 16-32×MIC (16-32 μg/ml), concentrations far higher than those achievable clinically. Although not determined according to NCCLS recommended procedures (National committee for Laboratory Standards), the inventors considered this the Minimal Bactericidal Concentration (MBC). INF 401 alone had no visible effect on S. aureus at concentrations up to 40 μg/ml. However, the MBC of erythromycin was potentiated at least 16 fold in the presence of 5 μg/ml of INF 401, a concentration at least 16 fold less than the MIC of this potentiator.

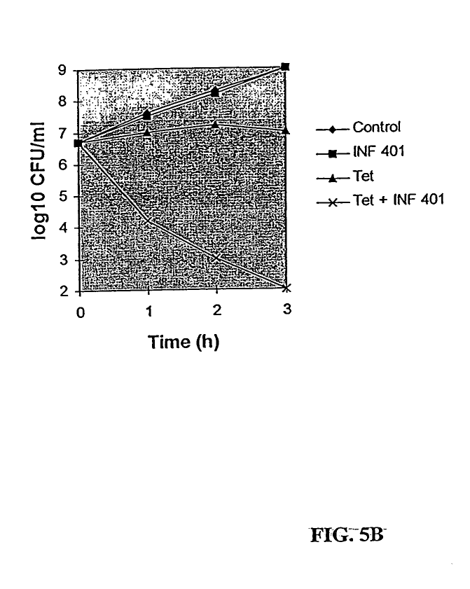

The bactericidal activity of INF 401 in combination with erythromycin is most strikingly evident in time kill studies. Logarithmically growing S. aureus (SA1199) were inoculated into tubes to OD₆₀₀ of 0.01 (6×10⁶ cells/ml) in the presence or absence of erythromycin (2×MIC) alone, or in combination with 5 μg/ml of INF 401, and incubated with shaking at 35° C. The initial inoculum number was determined by plating an appropriate dilution on LB agar plates and determining the number of colony forming units (CFUs). At different time points an aliquot was withdrawn from each tube and cell viability was determined by plating an appropriate dilution and counting CFUs after overnight incubation. Synergy was defined as at least a 100-fold decrease in CFUs by the combination as compared to the most active single agent (White et al., 1996). As shown in FIG. 4, INF 401 at a concentration of 5 μg/ml had no effect on the growth of S. aureus. Erythromycin alone, at 2×MIC, was bacteriostatic, completely inhibiting bacterial growth. However, the combination of INF 401 with erythromycin exhibited bactericidal activity with more than a 1000 fold decrease in viability in less than 3 h. The inventors considered this to be very rapid bactericidal activity since vancomycin, the antibiotic of choice for methicillin-resistant S. aureus (MRSA) infections, takes between 4-8 hours to decrease the viability of S. aureus by 1000-fold (Jeffrey et al., 1999).

### Example 3

### Potentiation of Other Inhibitors of Protein Synthesis

The inventors next evaluated whether INF 401 was bactericidal in combination with other protein synthesis inhibitors which are bacteriostatic to S. aureus. Checkerboard studies were performed with two-fold dilutions of each antibiotic in combination with INF 401 using the assay described above in Example 2. After overnight incubation the MIC of each antibiotic was determined by visualization of bacterial growth. Each antibiotic was tested at concentrations ranging from ¼×MIC to 8×MIC and INF 401 was tested at 11 concentrations ranging from 40 ng/ml to 20 μg/ml. Tetracycline, chloramphenicol and clindamycin were chosen since they represent three mechanistically different classes of antibiotics known to inhibit bacterial protein synthesis. The MBC of each antibiotic was determined as described in Example 2 in the absence or presence of INF 401.

TABLE 1
Effect of INF 401 on the MBCs of different bacteriostatic
antibiotics in S. aureus SAI199.

| Antibiotic | MIC | MBC^{a} | MBC^{b} |
|---|---|---|---|
| Erythromycin | 1 | 16-32 | 1 |
| Tetracycline | 0.5 | 8 | 0.25 |
| Chloramphenicol | 8 | >64 | 4 |
| Clindamycin | 0.25 | 4 | 0.125 |
| Rifampin | 0.009 | 0.06 | 0.0019 |
| Trimethroprim | 1 | 32 | 8 |

TABLE 1
Effect of INF 401 on the MBCs of different bacteriostatic
antibiotics in S. aureus SAI199.

| Antibiotic | MIC | MBC^{a} | MBC^{b} |
|---|---|---|---|
| Chlorhexidine | 1 | 4 | 1 |
| Cycloserine | 32 | >256 | 32 |
| Ethidium bromide | 16 | >128 | >128 |
| Ciprofloxacin | 0.5 | 0.5 | 0.5 |

MBC^{a} = MBC in the absence of INF 401.
MBC^{b} = MBC in the presence of 5 μg/ml of INF 401.

As shown in Table 1, erythromycin, tetracycline and chloramphenicol only demonstrated bactericidal effects on S. aureus at concentrations at least 16 fold higher than the MIC, concentrations which ae above those achievable clinically. Remarkably, however, INF 401 potentiated the activity of all three antibiotics being bactericidal, in combination, at concentrations equal to or less than the MIC of the antibiotic alone. This indicates that the combination of any one of these antibiotics with INF 401 would be bactericidal at clinically achievable concentrations. Clindamycin exhibited bactericidal activity at concentrations 4×MIC, a concentration at which S. aureus is considered to exhibit intermediate susceptibility (National Committee for Clinical Laboratory Standards, 1997). However, in the presence of INF 401, clindamycin was bactericidal at concentrations equal to the MIC, a four fold potentiation of bactericidal activity.

Interestingly, INF 401 did not potentiate the activity of either the fluoroquinolone ciprofloxacin, a bactericidal antibiotic which targets DNA replication, or ethidium bromide, a bacteriostatic agent which targets DNA synthesis, indicating that INF 401 may be specifically bactericidal to cells arrested in protein synthesis.

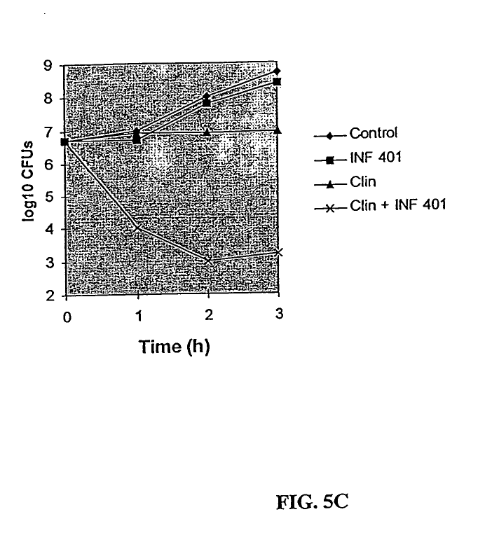

The inventors next confirmed that INF 401 was bactericidal in combination with chloramphenicol, tetracycline and clindamycin by performing time kill studies. As shown in FIG. 5, similar to studies with erythromycin, a 3 log₁₀ reduction in cell viability was observed in less than 3 h for all three antibiotics at a concentration of 1×MIC.

Additionally, INF 401 potentiated the bactericidal activity of the widely used antiseptic and antiplaque agent chlorhexidine by four fold.

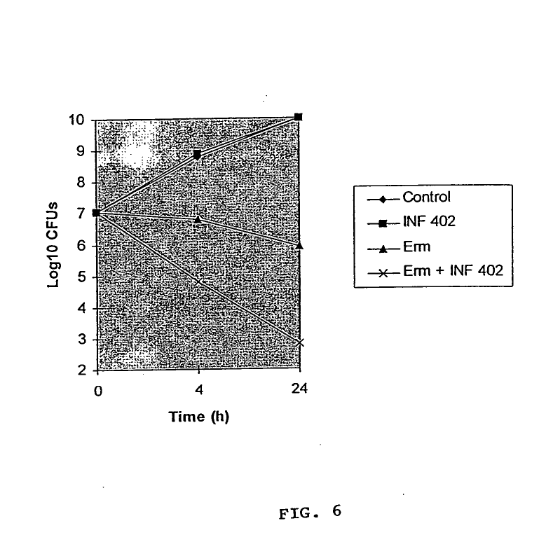

Table 2 and FIG. 6, demonstrate similar bactericidal results are obtained with INF 402.

TABLE 2
Effect of INF 402 on the MBCs of different
antibiotics in S. aureus SA1199.

| Antibiotic | MIC | MBC^{a} | MBC^{b} |
|---|---|---|---|
| Erythromycin | 1 | >8 | 1 |
| Tetracycline | 0.5 | >8 | 2 |
| Chloramphenicol | 8 | >64 | 8 |
| Clindamycin | 0.25 | 4 | 2 |
| Ethidium bromide | 16 | >128 | >128 |
| Ciprofloxacin | 0.5 | 0.5 | 0.5 |

MBC^{a} = MBC in the absence of INF 401.
MBC^{b} = MBC in the presence of 10 μg/ml of INF 402.

It would be desirable that an antibiotic potentiator of the present invention is active not only in S. aureus but also in other bacterial pathogens. The inventors have begun studies to investigate whether INF 401 can also potentiate the activity of translational inhibitors in another Gram positive pathogen, S. pneumoniae. It is well known that S. pneumoniae is more sensitive to protein synthesis inhibitors than S. aureus: chloramphenicol, tetracycline and erythromycin in the absence of INF 401 were bactericidal to S. pneumoniae at concentrations 2×MIC, 4×MIC and 1×MIC, respectively, whereas in S. aureus the same three antibiotics only showed bactericidal activity at concentrations of at least 8×MIC. However, in combination with INF 401, the bactericidal activity (MBC) of these three antibiotics was increased 4-16 fold so that a bactericidal effect was seen at concentrations well below those achievable clinically.

These data demonstrate that INF 401 exhibits rapid bactericidal activity in vitro in combination with clinically achievable concentrations of mechanistically different bacteriostatic antibiotics that target protein synthesis. Additionally, INF 401 also potentiates the activity of antibiotics which inhibit protein synthesis in S. pneumoniae, indicating that this compound may be active in a range of Gram positive and Gram negative pathogens, e.g., Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella and Campylobacter.

### Example 4

### INF 401 in Combination with Either Rifampin or Clindamycin Demonstrates Synergistic Activity In Vitro Against Biofilms of Staphylococcus aureus.

A major factor contributing to the difficulty in treating Gram positive pathogens such as Staphylococcus aureus, appears to be the ability of the bacteria to form biofilms in vivo. These microcolonies encased in extracellular polysaccharide material are inherently resistant to antibiotics, with the minimal concentration of an antibiotic to eradicate a biofilm (MBEC, Minimal Biofilm Eradicating Concentration) being far higher than the concentration required to inhibit the growth of planktonic cells. This inherent resistance is believed to contribute to the recurrence of chronic infections and the emergence of antibiotic resistance. The ability to increase the efficacy of antibiotics against biofilms could have tremendous implications in the treatment of device-related infections, endocarditis, osteomyelitis and infections in neutropenic individuals.

The inventors evaluated whether the lead compound, INF 401, which exhibits rapid bactericidal activity against planktonic S. aureus in combination with bacteriostatic concentrations of either chloramphenicol, tetracycline, erythromycin, clindamycin and, also, potentiates the bactericidal activity of rifampin, would potentiate the effect of these antibiotics against S. aureus biofilms. These studies were performed via the method of Dr. Howard Ceri (University of Calgary), who recently developed a method for the routine determination of the antibiotic susceptibility of biofilms. To date, the inventors have tested the effect of the combination of INF 401 on the ability of either clindamycin or rifampin to eradicate S. aureus biofilms in vitro.

S. aureus ATCC 29213 was selected for the study as previous studies of this isolate as a biofilm had been conducted and the isolate served as a quality assurance organism for NCCLS standardization of antibiotic testing. Biofilms were formed as previously described for this organism (Ceri et al., 1999) using the MBEC™ assay system. Checkerboard studies were performed with each antibiotic versus INF 401. Results are presented for each antibiotic in combination with the lowest concentration of INF 401 tested (0.3 μg/ml)

TABLE 3
Antibiotic susceptibility of S. aureus as a planktonic
population (MIC) and as a biofilm population (MBEC)

| | MIC, μg/ml | MBEC, μg/ml |
|---|---|---|
| Rifampin | 0.0075 | >2 |
| Rifampin + INF 401 | 0.0075 | 0.031 |
| Clindamycin | <1 | 256 |
| Clindamycin + INF 401 | <1 | <1 |

S. aureus ATCC 29213 as a planktonic population (MIC) and as a biofilm population (MBEC) in the presence or absence of INF 401 (0.3 μg/ml)

As shown in Table 3, the MBEC values (the concentration of antibiotic needed to eradicate the biofilm) were at least 256 fold higher than the MIC values for the antibiotics studied. In the presence of INF 401, there was a dramatic potentiation of the activity of both antibiotics against biofilms. In the case of rifampin, INF 401 at concentrations of 0.3 μg/ml lowered the MBEC values by 64 fold. Similarly, INF 401 dropped the MBEC values for clindamycin to the equivalent of the MIC value (at least a 256 fold potentiation). Since INF 401 alone has no effect on S. aureus growth at concentrations up to 20 μg/ml, the combination of this potentiator exhibits remarkable synergy with either antibiotic with respect to biofilm eradication (FIC index=<0.03 for rifampin and <0.019 for clindamycin).

Thus, the lead compound INF 401 exhibits remarkable synergistic activity in combination with either clindamycin or rifampin against S. aureus biofilms. This indicates that the combination of such a potentiator with any one of a number of antibiotics can be effective in eradicating chronic or recurring Gram positive infections and, furthermore, prevent the further emergence of antibiotic resistance.

### Example 5

### Testing INF 401 to be Bactericidal in Combination with Various Bacteriostatic Antibiotics

As described in Example 3, preliminary data demonstrates that INF 401 is bactericidal to S. aureus in combination with the bacteriostatic antibiotics erythromycin, tetracycline, chloramphenicol and clindamycin, indicating that INF 401 may potentiate the activity of a broad spectrum of bacteriostatic antibiotics. It will be valuable in the present invention to determine whether INF 401 can potentiate other inhibitors of protein synthesis such as aminoglycosides, other macrolides and tetracyclines, in addition to newer classes of bacteriostatic antibiotics such as oxazolidinones and ketolides which are currently in development. The following antibiotics will be obtained for testing: the aminoglycosides tobramycin, kanamycin, gentamycin, streptomycin; tetracycline derivatives including doxycycline and GAR-936 (Wyeth-Ayerst, NJ); the macrolides clarithromycin (Abbot Laboratories, Abbot Park, Ill.) and azithromycin (Pfizer Central Research, Groton, Conn.); the ketolide (HMR-3647) telithromycin (Hoechst Marion Roussel, Paris, France); and the oxazolidinone linezolid (Pharmacia Upjohn, Kalamazoo, Mich.). Additionally, other antibiotics which do not target protein synthesis will be tested since this may help gain insight into the mechanism of action of INF 401.

The inventors will first determine the effect of INF 401 on the MBC of each of the antibiotics by performing checkerboard microdilution studies as described in Example 2. Briefly, using the S. aureus strain SA1199, each of the antibiotics will be tested at concentrations ranging from ¼×MIC to 8×MIC vs. different concentrations of INF 401. After incubation, 2 μl of the resuspended cells will be transferred to 150 μl of fresh medium and examined for growth after 24 h incubation at 35° C. Since the inventors have demonstrated that INF 401 is rapidly bactericidal in combination with either erythromycin, chloramphenicol or tetracycline, with a greater than 1000-fold decrease in viable cell number by 3 hours, the cells will be incubated for 6 hours instead of overnight before transfer. Subsequently, bactericidal combinations in S. aureus will be confirmed by time kill studies as described in Example 2. The optimal concentration of the antibiotic and INF 401 for these studies will be determined from MBC checkerboard titrations.

### Example 6

### Evaluating INF 401 Activity in Combination with Bacteriostatic Antibiotics Against a Range of Gram Positive Pathogens

In preliminary data the inventors have shown that INF 401 potentiates the activity of chloramphenicol, tetracycline and erythromycin in S. aureus and S. pneumoniae. Here the inventors will determine whether INF 401 potentiates the activity of these bacteriostatic antibiotics and others identified in Example 5, against other species of bacteria including both Gram positive and Gram negative pathogens. The bacterial strains will be obtained from ATCC and their sensitivity to a bacteriostatic antibiotic alone, or in combination with INF 401, will be determined by broth microdilution assays as described in Example 2 using media and conditions as recommended by NCCLS.

One possible limitation to the combination of INF 401 with a bacteriostatic antibiotic is that the combination may be ineffective in strains resistant to the antibiotic alone. If, for example, the combination of a macrolide such as erythromycin with INF 401 was ineffective in macrolide-resistant strains then this would limit the use of the combination to less than 40% of S. aureus isolates which remain sensitive to erythromycin (Pfaller et al., 1998). In order to predict possible applications of the envisioned combination drug it will be important to assess the effectiveness of the combination in vitro against antibiotic-resistant isolates. To this end the combination of INF 401 and a given bacteriostatic antibiotic will be tested in strains of S. aureus expressing different resistant mechanisms to the antibiotic. For example, macrolide-resistant strains with target site alterations of the ribosome (ermA, B or C), a mechanism which account for almost 90% of resistance to macrolides, lincosamides and streptogramins Lina et al., 1999), and strains expressing the mrsA macrolide efflux determinant will be tested.

Another potential limitation to the envisioned combination drug is the possibility of resistance developing to either component. Such a situation has been observed for bacteria which, through mutations in the β-lactamase gene, have developed resistance to Augmentin (a combination of ampicillin and clavulanic acid, an inhibitor of β-lactamase). Similarly mutations in the potential target of INF 401, or the expression of systems leading to the modification and inactivation of this compound or, alternatively, the antibiotic used in combination, could result in the emergence of resistance. Here the inventors will evaluate the ability of S. aureus to develop resistance to the combination of INF 401 and a given antibiotic in vitro.

The inventors have attempted to isolate resistant mutants by selecting approximately 10¹⁰ logarithmically growing S. aureus on solid agar containing either chloramphenicol alone or a bactericidal combination of chloramphenicol and INF 401, at two and four fold higher than the MBC (Markham and Neyfakh, 1996). No mutants were obtained indicating that it may be difficult for S. aureus to develop resistance to the potentiator: antibiotic combination. Resistant mutants were obtained only after chemical mutagenesis of S. aureus prior to selection (see Example 7).

### Example 7

### Molecular Mechanism of Action of INF 401

Although INF 401 and its analogs demonstrate a novel and rather striking biological effect, its molecular mechanism of action is not completely understood. An understanding of this mechanism, while of tremendous interest from the scientific standpoint, would clearly benefit the future clinical use of such compounds and their approval by regulatory agencies. The inventors have begun to identify the key principles underlying the mechanism of action of INF 401 and antibiotics in the present invention.

### Characterization of the Process of Bacterial Killing Mediated by the Combination of Antibiotics with INF 401

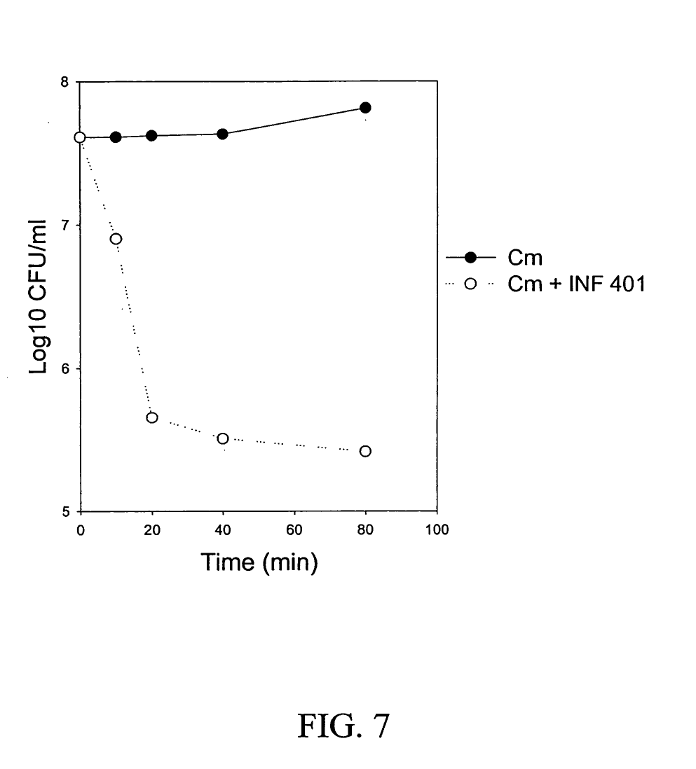

In order to investigate the mechanism of killing mediated by INF 401 in combination with antibiotics, the inventors first looked at the effect of this compound on cellular morphology, protein and DNA. S. aureus strain ATTC 29213 was inoculated at OD₆₀₀ 0.02 in LB supplemented with a bacteriostatic concentration of chloramphenicol (20 μg/ml), in the absence or presence of 2 μg/ml INF 401, and analyzed after incubation for 10, 20, 40, and 80 min. Although the majority of cells incubated in the presence of INF 401 and chloramphenicol were dead in the first 20 min (FIG. 7), surprisingly no obvious morphological differences were observed microscopically at any time point. Likewise, there were no differences observed in either the amount or pattern of cellular protein among treated cells. However, the analysis of the DNA isolated from cells demonstrated that, while chloramphenicol or INF 401 alone did not affect DNA integrity, their combination resulted in the rapid degradation of DNA (not shown).

### INF 401 Activity is Iron-Dependent

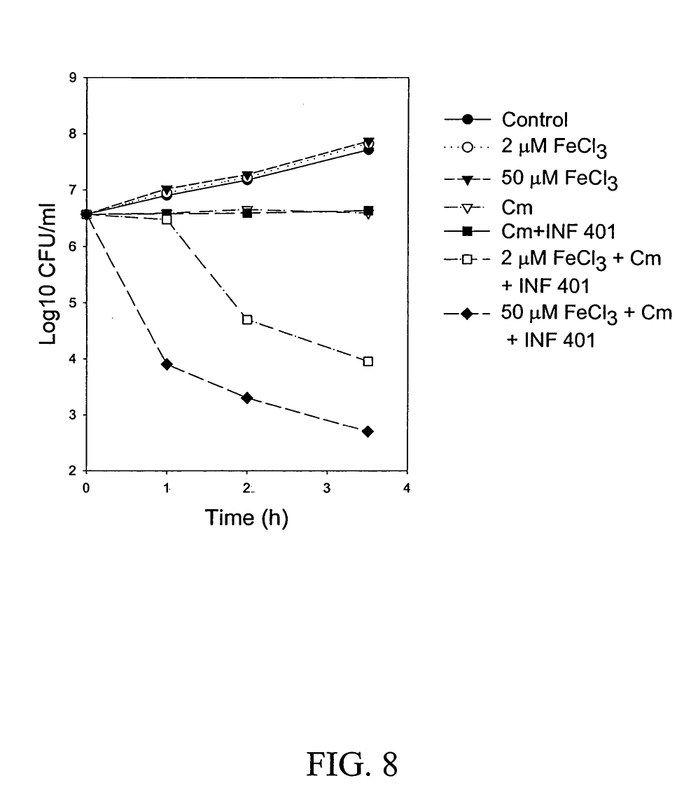

Analysis of the chemical structure of INF 401 suggested that this compound, as well as other classes of active compounds obtained in the screening, had metal chelating properties. Indeed, it appeares that iron was required for its bactericidal activity since the iron chelators deferoxamine and EDDHA were shown to completely ablate killing and this effect was reversed by the addition of exogenous Fe³⁺ (not shown). Likewise, INF 401 did not potentiate the bactericidal activity of chloramphenicol in an iron-restricted siderophore detection media (SSD, Heinrichs, 1999) while supplementation of the medium with 2 μM FeCl, restored the ability of INF 401/chloramphenicol to kill cells. An increase of FeCl, concentration to 50 μM resulted in a further increase of INF 401 activity (FIG. 8).

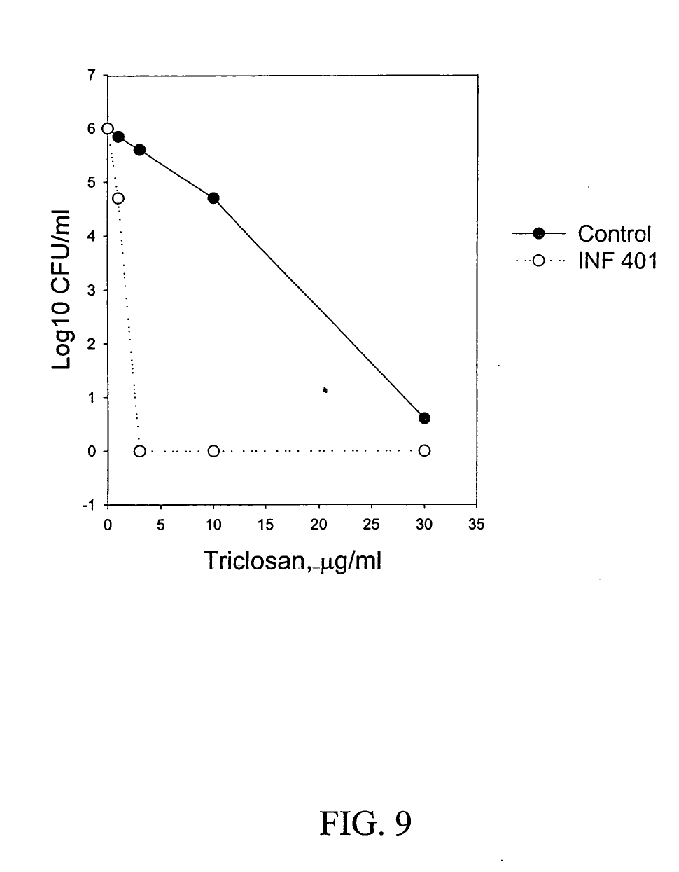

Similar results were seen in LB medium: an increase in the iron concentration of LB by supplementation with 50 μM FeCl₃ increased the bactericidal activity of [NF 401 in the presence of chloramphenicol by one order of magnitude while having no effect on chloramphenicol alone. In the same iron-supplemented media, INF 401 was seen to dramatically potentiate the bactericidal activity of the antiseptic agent triclosan against S. aureus (FIG. 9).

Inventors also investigated whether transition metals other than iron can facilitate the bactericidal activity of INF 401. Supplementation of LB with 50 μM CuSO₄ did not affect viability of cells incubated with or without 20 μg/ml of chloramphenicol. However, similar to FeCl₃ supplementation, the combination of chloramphenicol with 2 μg/ml INF 401 in the presence of CuSO₄ resulted in more than a ten-fold decrease in cell viability compared to cells incubated without CuSO₄,

### INF 401 Transports Iron Inside the Cell

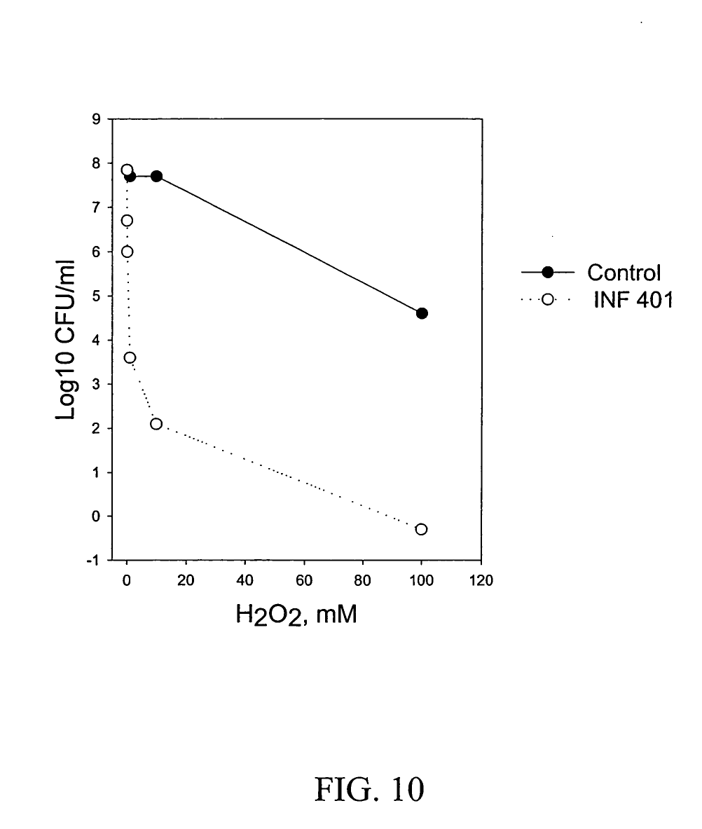

The results demonstrate that cell death caused by INF 401 is iron-dependent and associated with severe DNA damage. Therefore, inventors speculated that INF 401 is involved in the Fenton reaction, a reaction that involves the iron-dependent conversion of superoxide anions and hydrogen peroxide to reactive oxygen species resulting in damage to DNA and other molecules. Indeed, INF 401 was shown to dramatically potentiate the bactericidal activity of hydrogen peroxide against S. aureus FIG. 10).

The effect of INF 401 was evaluated in an in vitro Fenton reaction. The plasmid pUC18 was incubated with 1 nM hydrogen peroxide and various concentrations of FeSO₄ which resulted in nicking of the plasmid and disappearance of the supercoiled form. Addition of 2 μg/ml of INF 401 to the reaction did not result in any obvious changes in either the pattern or time course of DNA degradation indicating that this compound does not directly catalyse the Fenton reaction.

Next, investigated was whether INF 401 affects intracellular iron accumulation. An ⁵⁵Fe transport assay (modified from Sebulsky, 2000) was used to examine the ability of INF 401 to transport iron inside the cell. Briefly, exponentially growing cells were diluted to OD₆₀₀ 0.1 in LB, incubated with ⁵⁵FeCl₃ for 30 min at 37° C., washed and counted in scintillation fluid using the tritium channel of a scintillation system. Comparison of the amount of ⁵⁵Fe accumulated by cells cultured with or without chloramphenicol, demonstrated that the antibiotic alone did not affect iron accumulation (4,976 vs 9,232 DPM). However, the presence of INF 401 in either the absence or presence of antibiotic led to a dramatic increase in iron accumulation (209,215 and 231,621 DPM, respectively).

Additionally, the bactericidal activity of a series of active and inactive pairs of analogues of INF 401, which differed by only a small alteration in the chemical structure, correlated with their ability to cause accumulation of intracellular iron in S. aureus (Table 4). Activity of the compound is depicted as the minimal concentration required to kill 99.9% of S. aureus in the presence of 4 μg/ml erythromycin. Assuming that these slight alterations in chemical structure do not affect either the ability of the analog to bind iron, or its transport into the cell, these data support the idea that INF 401 damages DNA by creating an excess of intracellular iron.

TABLE 4
Accumulation (Accm) of ⁵⁵Fe (DPM) in S. aureus SA 29213 in the presence or absence of
active and inactive analogues of INF 401 at a concentration of 2 μg/ml.

| | | ⁵⁵Fe | | | ⁵⁵Fe |
|---|---|---|---|---|---|
| | Activity, | Accm., | | Activity, | Accm., |
| Chemical structure | μg/ml | DPM | Chemical structure | μg/ml | DPM |
| Control | 0 | 511 | 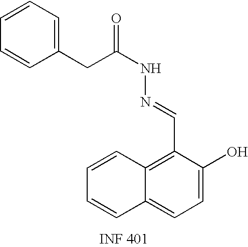 | 0.15 | 36,941 |
| 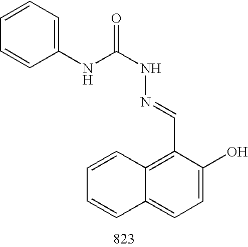 | 0.3 | 14,400 | 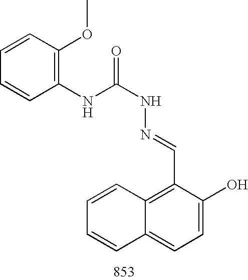 | >10 | 1,900 |
| 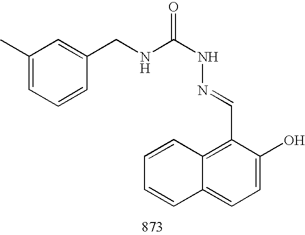 | >10 | 1,500 | 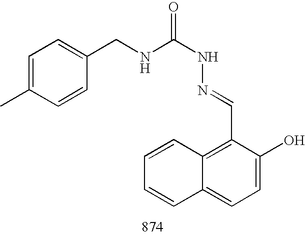 | 1.25 | 9,100 |
| 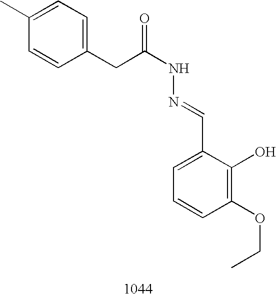 | >10 | 1,200 | 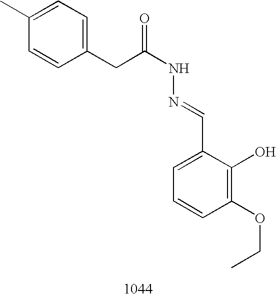 | 1.25 | 17,400 |

### Cells Protect Themselves Against INF 401 by the Oxidative Stress Response

Considering the fact that INF 401 exhibits bactericidal activity only in the presence of antibiotic, inventors propose that antibiotic and INF 401 would act synergistically to increase iron accumulation. However, as described earlier, INF 401 alone promoted the accumulation of iron to the same extent as INF 401 with antibiotic although INF 401 has no effect on Staphylococcal growth. This suggested that Staphylococci can normally tolerate this level of intracellular iron, possibly by the induction of a protective response. In support of this hypothesis, exposure of S. aureus to INF 401 led to the increased expression of two proteins, 20 and 24 kDA, as studied by metabolic labeling. In parallel, inventors began attempts to identify INF 401-resistant mutants. Since no spontaneous mutants could be selected for resistance to INF 401 in combination with chloramphenicol, cells were randomly mutagenized with ethylmethane sulfonate (EMS) prior to selection. One of three clones obtained was shown to have several proteins constitutively overexpressed that exactly matched the proteins induced in response to INF 401 exposure. The most abundant of these proteins proved to be alkyl hydroperoxide reductase, AhpC, a protein that protects cells from the toxic effects of oxidants by directly eliminating them.

### Example 8

### Testing the Toxicity of the Identified Potentiators for Human Cell Lines

In order to be used in clinics, the antibiotic potentiators of the present invention should lack any toxicity for humans. In the present invention, the inventors will determine the toxicity of identified potentiators for a human cell line cultivated in vitro, which will serve as a good indicator for eliminating toxic potentiator leads. The toxicity of each potentiator to HeLa cells will be determined in a 96 well assay. Cells will be cultivated for 72 h with different concentrations of each compound and the level of growth inhibition will be determined by an MTS assay using the Cell Titer 96 Aqᵤₑₒᵤₛ assay (Promega) from which the IC₅₀ will be determined (cell Titer 96).

From these toxicity studies the inventors will calculate a selectivity index for each potentiator which will be the IC₅₀ of the potentiator for HeLa cells/minimal concentration of the potentiator with bactericidal activity in combination with erythromycin at 4×MIC. Only those potentiators whose effectiveness are at least ten times smaller than the IC₅₀ in the cytotoxicity assay will be considered for further development.

Based on these results the following mechanism of action of INF 401 seems plausible: INF 401 facilitates the accumulation of iron inside the cell thus leading to increased levels of reactive oxygen species. As a defense mechanism against oxidative stress, the expression of several antioxidant proteins is induced. However, in the presence of antibiotics affecting protein synthesis, activation of this defense mechanism is inhibited and oxidative stress leads to cell death.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,448,962
U.S. Pat. No. 4,499,091
U.S. Pat. No. 4,668,784
U.S. Pat. No. 4,704,459
U.S. Pat. No. 4,795,751
U.S. Pat. No. 5,532,239
DE Patent Number 3,142,854
EP Number 206283
Ahmed et al., “Mutants of the Bacillus subtilis multidrug transporter Bmr with altered sensitivity to the antihypertensive alkaloid reserpine,” J. Biol. Chem., 268:11086-11089, 1993.
Baranova, Danchin, Neyfakh, “Mta, a global Merit-type regulator of the Bacillus subtilis multidrug-efflux transporters.,” Molecular Microbiology, 31:1549-1560, 1999.
Cell Titer 96, “Non-radioactive cell proliferation technical bulletin, #245,” Promega Corp. 1998.
Center for Disease Control, “Defining the public health impact of drug-resistant Streptococcus pneumonia; report of a working group,” MMWR, 45:1-20, 1996.
Challis and Challis “Amides and Related Compounds,” In: Comprehensive Organic Chemistry” Barton, Ollis, Pergamon (eds), Oxford, 2(9.9),1979
Cormican and Jones, “Emerging resistance to antimicrobial agents in Gram positive bacteria,” Drugs, 51(1):6-12, 1996.
Costerton et al., “Bacterial Biofilms: a common cause of persistent infections,” Science, 284:1318-1322, 1999.
Dyson, Barnes, Harrison, “Infective endocarditis: an epidemiological review of 128 episodes,” J. Infect., 38(2):87-93, 1999.
Heinrichs J H, Gatlin L E, Kunsch C, Choi G H, Hanson M S. 1999. Identification and characterization of SirA, an iron-regulated protein from Staphylococcus aureus. J. Bacteriol. 181(5):1436-43.
Kaatz, et al., “Mechanisms of fluoroquinolone resistance in Staphylococcus aureus,” J. Infect. Dis., 163:1080-1086, 1990.
Karwowska, Davies, Jadavji, “Epidemiology and outcome of osteomyelitis in the era of sequential intravenous-oral therapy,” Pediatr. Infect Dis., 17:1021-1026, 1998.
Koenig and Kaye, “Enterococcal endocarditis,” New Engl. J. Med., 264:257, 1961.
Koh, L. L., 0. L. Kon, et al. (1998). “Complexes of salicylaldehyde acylhydrazones: Cytotoxicity, QSAR and cyrstal structure of the sterically hindered t-butyl dimer.” J. Inorg. Biochem. 72: 155-162.
Korten et al., “Analysis by PCR and direct sequencing of gyrA mutations associated with fluoroquinolone resistance in Enterococcus faecalis,” Antimicrob. Agents Chemother., 38:2091-2094, 1994.
Lina, Quaglia, Reverdy, Leclercq, Vandenesch, Etienne, “Distribution of Genes Encoding Resistance to Macrolides, Lincosamides, and Streptogramins among Staphylococci,” Antimicrob. Agents Chemother., 43:1062-1066, 1999.
Lovejoy, D. B. and D. Richardson (2000). Structure-Activity Relationships of Iron Chelators of the Pyridoxal Isonicotinoyl Hydrazone Class. On-line Proceedings of the 6th Internet World Congress for Biomedical Sciences (INABIS 2000) at Castilla La Mancha University, Spain (available from URL: http://www.uclm.es/-inabis2000/symposia/files/145/index.htm).
Markham and Neyfakh, “inhibition of the multidrug transporter NorA prevents emergence of norfloxacin resistance in Staphylococcus aureus,” Antimicrob. Agents Chemother., 40:2673-2674, 1996.
Markham, Westhaus, Klyachko, Johnson, Neyfakh, “Multiple novel inhibitors of the NorA multidrug transporter of Staphylococcus aureus,” Antimicrob. Agents and Chemother., 43:2404-2408, 1999.
Moellering, Wennersten, Weinberg, “Studies on antibiotic synergism against enterococci, J. Lab. Clin. Med., 77:821-828, 1971.
National Committee for Clinical Laboratory Standards, Methods for dilution antimicrobial susceptibility tests for bacteria that grow aerobically—fourth edition, Approved standard M7-A4. National Committee for Clinical Laboratory Standards, Villanova, Pa., 1997.
National Committee for Laboratory standards, Methods for determining bactericidal activity of antimicrobial agents. Approved guideline, Document M26-A. National Committee for Laboratory Standards, Villanova, Pa. In Press.
Novick, “The Staphylococcus as a molecular genetic system,” In: Molecular biology of the Staphylococci, (ed) Novick, VCH Publishers, NY, p. 1-37, 1990.
Olanow C W, Arendash G W. “Metals and free radicals in neurodegeneration.” Curr Opin Neurol 1994; 7:548-58.
Peterson and Shanholtzer, “Tests for bactericidal effects of antimicrobial agents: Technical performance and clinical relevance,” Clin. Microbiol. Rev., 5:420-432, 1992.
Pfaller, Jones, Doern, Kugler and The Sentry participants Group, “Bacterial pathogens isolated from patients with bloodstream infection: Frequencies of occurrence and antimicrobial susceptibility patterns from the SENTRY antimicrobial surveillance program,” Antimicrob. Agents and Chemother., 42:1762-1770, 1998.
Ranford, J. D., Vittal, J. J., and Wang, Y. M. (1998). “Dicopper(II) Complexes of the Antitumor Analogues Acylbis(salicylaldehyde hydrazones) and Crystal Structures of Monomeric [Cu₂(1,3-propanedioyl bis(salicylaldehyde hydrazone))(H₂O)₂](ClO₄)₂ 3H₂O) and Polymeric [{Cu₂(1,6-hexanedioyl bis(salicylaldehyde hydrazone))(C₂H₅OH)₂}ₘ](ClO₄)₂ₘ m(C₂H₅OH).” Inorg. Chem. 37(6): 1226-1231.
Richardson, D. R. and P. V. Bernhardt (1999). “Crystal and molecular structure of 2-hydroxy-1-naphthaldehyde isonicotinoyl hydrazone (NIH) and its iron(II) complex: an iron chelator with anti-tumor activity.” J. Biol. Inorg. Chem. 4(3): 266-273.
Saccente and Cobbs, “Diagnosis and management of infective carditis: Clinical approach to infective endocarditis,” Cardiology Clinics, 14:352-363, 1996.
Sandstrom, J., Dynamic NMR Spectroscopy, Academic Press, London, p. 96, 1982.
Schierholz, Beuth, Pulverer, “Killing effects of antibiotics and two-fold antimicrobial combinations on proliferating and non growing staphylococci,” Zentralbl Bakteriol, 288:527-39, 1998.
Sebulsky M T, Hohnstein D, Hunter M D, Heinrichs D E. 2000. Identification and characterization of a membrane permease involved in iron-hydroxamate transport in Staphylococcus aureus. J. Bacteriol. 182(16):4394-400.
Sohnenshein, Hoch, Losick, Staphylococcus, Novick, In: Bacillus subtilis and other Gram-positive bacteria, ASM Press, Washington D.C., pp. 17-34, 1993.
Stickler, “Biofilms,” Curr. Opin. Microbiol., 2: 270-275, 1999.
Tennant, “Imines, Nitrones, Nitriles and Isocyanides” In: Comprehensive Organic Chemistry, Barton, Ollis, Pergamon (Eds), Oxford, 2(8), 1979.
Watson, Clements, Foster, “Characterization of the starvation-survival response in Staphylococcus aureus,” J. Bacteriol., 180:1750-1758, 1998.
White, Burgess, Manduru, Bosso, “Comparison of three different in vitro methods of detecting synergy: Time-kill, checkerboard and E-test,” Antimicrob. Agents Chemother., 40:1914-1918, 1996.
Zhou, Martin, Johnson, “Multiple binding modes of flavones as protein tyrosine kinase inhibitors,” J. Computer-Aided Molec. Design.

1. A method for increasing the bactericidal action of an antibacterial agent comprising contacting a bacterium with an antibiotic potentiator, wherein said potentiator is an acyl hydrazide, an oxy amide, or an 8-hydroxy quinoline.

2. The method of claim 1, wherein said acyl hydrazide has the general formula: 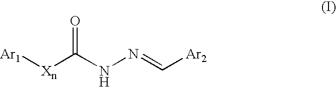
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1.

3. The method of claim 2, wherein Ar₁ is selected from the group consisting of phenyl-, 4-toluoyl-, 4-isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo(2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo[2.2.1]heptane.

4. The method of claim 2, wherein Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl.

5. The method of claim 1, wherein said acyl hydrazide has the formula: 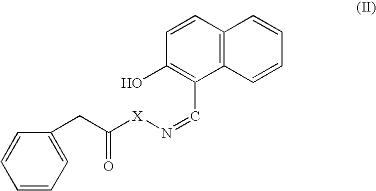
wherein X is CHI, C(CH₃)₂, NH, N-alkyl or N-phenyl.

6. The method of claim 1, wherein said acyl hydrazide has the formula: 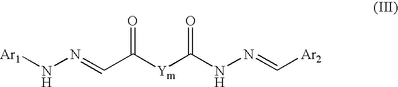
wherein Ar₁ and Ar₂ are independently aryl or substituted aryls, Y comprises one or more of C, N, and O and m is 1, 2, 3, 4, 5, 6, 7 or 8.

7. The method of claim 6, wherein Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-, 4,6-dimethoxy-2-phenol-, 4-methoxy-2-phenol and 2-thio-1-phenyl.

8. The method of claim 1, wherein said oxy amide has the formula: 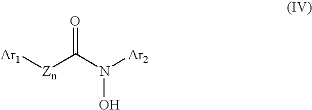
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1.

9. The method of claim 8, wherein Ar₁ is an anisole, n=0, and Ar₂ is a phenyl.

10. The method of claim 1, wherein said 8-hydroxyquinoline has the formula: 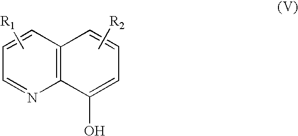
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

11. The method of claim 10, wherein R₁ is 2-(3,5-dimethyl-pyrazol-1-yl) and R₂ is H.

12. The method of claim 1, wherein said bacterium is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

13. The method of claim 1, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

14. A method of treating a subject with a bacterial infection comprising administering to said subject an antibacterial agent and an antibiotic potentiator, wherein said potentiator is an acyl hydrazide, an oxy amide, or an 8-hydroxy quinoline.

15. The method of claim 14, wherein said acyl hydrazide has the general formula: 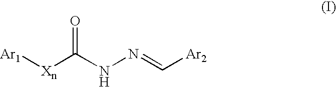
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl or S and n is 0 or 1.

16. The method of claim 15, wherein Ar₁ is selected from the group consisting of phenyl-, 4-toluoyl-, 4-isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo[2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo[2.2.1]heptane.

17. The method of claim 15, wherein Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl.

18. The method of claim 14, wherein said acyl hydrazide has the formula: 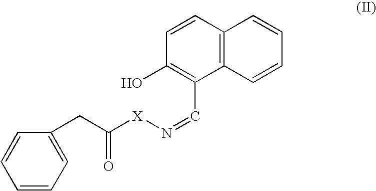
wherein X is CH₂, C(CH₃)₂, NH, N-alkyl or N-phenyl.

19. The method of claim 14, wherein said acyl hydrazide has the formula: 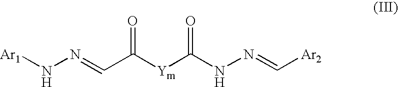
wherein Ar₁ and Ar₂ are independently aryl or substituted aryls, Y comprises one or more of C, N, and O and m is 1, 2, 3, 4, 5, 6, 7 or 8.

20. The method of claim 19, wherein Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-, 4,6-dimethoxy-2-phenol-, 4-methoxy-2-phenol and 2-thio-1-phenyl.

21. The method of claim 14, wherein said oxy amide has the formula: 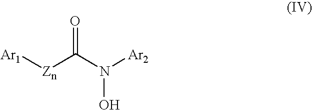
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1.

22. The method of claim 21, wherein Ar₁ is an anisole, n=, and Ar₂ is a phenyl.

23. The method of claim 14, wherein said 8-hydroxyquinoline has the formula: 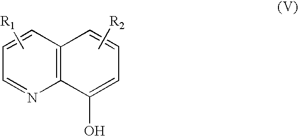
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

24. The method of claim 23, wherein R₁ is 2(-3,5-dimethyl-pyrazol-1-yl) and R₂ is H.

25. The method of claim 14, wherein said bacterial infection is of the genus Staphylococcus, Streptococcus, Enterococcus, Mycobacterium, Listeria, Pseudomonas, Serratia, Escherichia, Klebsiella, Haemophilus, Enterobacter, Proteus. Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

26. The method of claim 14, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

27. The method of claim 26, further comprising a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein said first and said second antibacterial agents are chemically distinct compounds.

28. A bactericidal pharmaceutical composition comprising an antibacterial agent and an antibiotic potentiator, wherein said potentiator is an acyl hydrazide, an oxy amide, or an 8-hydroxy quinoline.

29. The composition of claim 28, wherein said acyl hydrazide has the general formula: 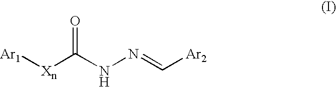
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1.

30. The composition of claim 29, wherein Ar₁ is selected from the group consisting of phenyl-, 4-toluoyl-, 4-isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo[2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo[2.2.1]heptane.

31. The composition of claim 29, wherein Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl.

32. The composition of claim 28, wherein said acyl hydrazide has the formula: 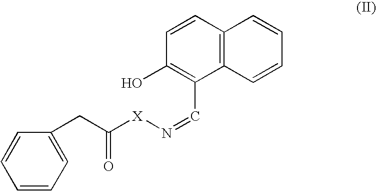
wherein X is CH₂, C(CH₃)₂, NH, N-alkyl or N-phenyl.

33. The composition of claim 28, wherein said acyl hydrazide has the formula: 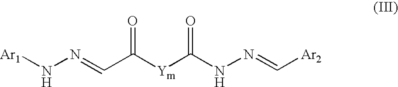
wherein Ar₁ and Ar₂ are independently aryl or substituted aryls, Y comprises one or more of C, N, and O and m is 1, 2, 3, 4, 5, 6, 7 or 8.

34. The composition of claim 33, wherein Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, S-bromo-3-methoxy-2-phenol, 3-ethoxy-2-phenol-, 4,6-dimethoxy-2-phenol-, 4-methoxy-2-phenol and 2-thio-1-phenyl.

35. The composition of claim 28, wherein said oxy amide has the formula: 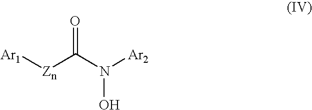
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1.

36. The composition of claim 35, wherein Ar₁ is an anisole, n=0, and Ar₂ is a phenyl.

37. The composition of claim 28, wherein said 8-hydroxyquinoline has the formula: 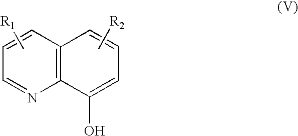
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

38. The composition of claim 37, wherein R₁ is 2-(3,5-dimethyl-pyrazol-1-yl) and R₂ is H.

39. The bactericidal composition of claim 28, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

40. The bactericidal composition of claim 39, further comprising a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein said first and said second antibacterial agents are chemically distinct compounds.

41. A method of screening for candidate acyl hydrazide antibiotic potentiators, oxy amide antibiotic potentiators or 8-hydroxy quinoline potentiators comprising:
(a) contacting a bacterial cell with an antibacterial agent;
(b) contacting a bacterial cell with said antibacterial agent and an acyl hydrazide, an oxy amide, or an 8-hydroxy quinoline; and
(c) comparing the bactericidal effect of said antibacterial agent in the presence and absence of said acyl hydrazide, oxy amide or 8-hydroxy quinoline,
wherein a decrease in bacterial cell viability indicates said candidate acyl hydrazide, oxy amide or 8-hydroxy quinoline is an antibiotic potentiator.

42. The method of claim 41, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

43. The method of claim 41, wherein said bacterial cell is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

44. A method of treating a subject for a bacterial biofilm infection comprising administering to said subject an antibacterial agent and an antibiotic potentiator, wherein said potentiator is an acyl hydrazide, an oxy amide, or an 8-hydroxy quinoline.

45. The method of claim 44, wherein said acyl hydrazide has the general formula: 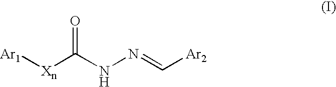
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or I.

46. The method of claim 45, wherein Ar₁ is selected from the group consisting of phenyl-, 4-toluoyl-, 4 isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo[2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo(2.2.1]heptane.

47. The method of claim 45, wherein Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl.

48. The method of claim 44, wherein said acyl hydrazide has the formula: 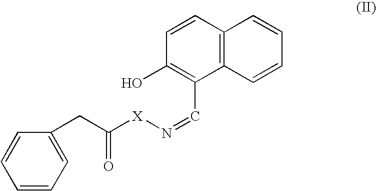
wherein X is CH₂, C(CH₃)₂, NH, N-alkyl or N-phenyl.

49. The method of claim 44, wherein said acyl hydrazide has the formula: 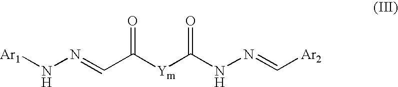
wherein Ar₁ and Ar₂ are independently aryl or substituted aryls, Y comprises one or more of C, N, and O and m is 1, 2, 3, 4, 5, 6, 7 or 8.

50. The method of claim 49, wherein Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-, 4,6-dimethoxy-2-phenol-, 4-methoxy-2-phenol and 2-thio-1-phenyl.

51. The method of claim 44, wherein said oxy amide has the formula: 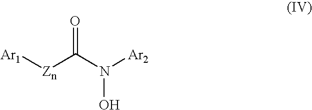
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1.

52. The method of claim 51, wherein Ar₁ is an anisole, n=0, and Ar₂ is a phenyl.

53. The method of claim 44, wherein said 8-hydroxyquinoline has the formula: 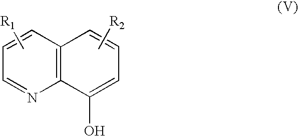
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

54. The method of claim 53, wherein R₁ is 2-(3,5-dimethyl-pyrazol-1-yl) and R₂ is H.

55. The method of claim 44, wherein said biofilm is of the genus Staphylococcus, Streptococcus, Enterococcus, Mycobacterium, Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

56. The method of claim 52, wherein said infection is resistant to antibacterial agents.

57. The method of claim 56, wherein said infection is a chronic infection or persistent infection.

58. The method of claim 54, wherein said infection is endocarditis, osteomyelitis, an infection in a neutropenic subject or a biomaterial infection.

59. The method of claim 44, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

60. The method of claim 59, further comprising a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein said first and said second antibacterial agents are chemically distinct compounds.

61. A pharmaceutical composition for inhibiting bacterial biofilm viability comprising an antibacterial agent and an antibiotic potentiator, wherein said potentiator is an acyl hydrazide, an oxy amide, or an 8-hydroxy quinoline.

62. The composition of claim 61, wherein said acyl hydrazide has the general formula: 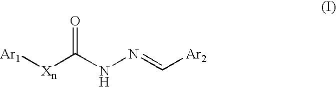
wherein Ar₁ and Ar₂ are independently aryl, substituted aryl, cycloalkyl, bicycloalkyl, substituted bicycloalkyls, bicycloalkenyl, or substituted bicycloalkenyl, X is CH₂, C(CH₃)₂, NH, N-alkyl, N-phenyl, or S and n is 0 or 1.

63. The composition of claim 62, wherein Ar₁ is selected from the group consisting of phenyl-, 4-toluoyl-, 4-isopropyl-1-phenyl-, 4-t-butyl-1-phenyl-, 2-anisole, 4-ethyl-1-phenyl-, 3-chloro-1-phenyl-, bicyclo[2.2.1]heptane, bicyclo[2.2.1]hept-5-ene, bicyclo[4.1.0]heptane, hexahydro-2,5-methano-pentalene, 1-pyridin-3-yl-, 7,7,-dimethyl-2-oxo-bicyclo[2.2.1]heptane, cylcohexyl-, cycloheptyl- and 4,7,7-trimethyl-3-oxo-2-oxa-bicyclo[2.2.1]heptane.

64. The composition of claim 62, wherein Ar₂ is selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-4,6-dimethoxy-2-phenol-, 4-methoxy-2-phenol and 2-thio-1-phenyl.

65. The composition of claim 61, wherein said acyl hydrazide has the formula: 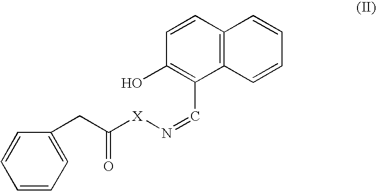
wherein X is CH₂, C(CH₃)₂, NH, N-alkyl or N-phenyl.

66. The composition of claim 61, wherein said acyl hydrazide has the formula: 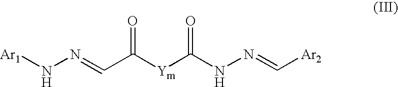
wherein Ar₁ and Ar₂ are independently aryl or substituted aryls, Y comprises one or more of C, N, and O and m is 1, 2, 3, 4, 5, 6, 7 or 8.

67. The composition of claim 66, wherein Ar₁ and Ar₂ are independently selected from the group consisting of 2-hydroxy-1-naphthyl-, 2-phenol-, 3,5-dichloro-2-phenol-, 4-diethylamino-2-phenol-, 3-methyl-2-phenol-, 4-methyl-2-phenol, 5-methyl-2-phenol, 5-bromo-2-phenol, 5-bromo-3-methoxy-2-phenol, 3-ethoxy-2-phenol-, 4,6-dimethoxy-2-phenol-, 4-methoxy-2-phenol and 2-thio-1-phenyl.

68. The composition of claim 61, wherein said oxy amide has the formula: 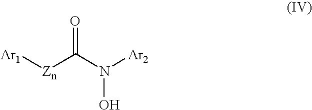
wherein Ar₁ and Ar₂ are independently phenyl, naphthyl, toluoyl, anisole, alkylphenyl, alkoxyphenyl, halophenyl, benzyl, or pyridinyl, and Z comprises one or more of C, N, and O, and n=0 or 1.

69. The composition of claim 68, wherein Ar₁ is an anisole, n=0, and Ar₂ is a phenyl.

70. The composition of claim 61, wherein said 8-hydroxyquinoline has the formula: 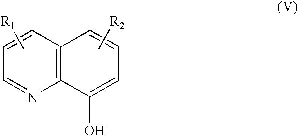
wherein R₁ and R₂ are independently H, alkyl, alkoxy, a halogen, substituted or unsubstituted 1-allylphenyl, benzyl, a hydrazino group (—NHNH2), a substituted hydrazino group, pyrazolyl, alkyl substituted pyrazolyl, an unsubstituted pyridazinyl group, or a substituted pyridazinyl group.

71. The composition of claim 70, wherein R, is 2-(3,5-dimethyl-pyrazol-1-yl) and R₂ is H.

72. The composition of claim 61, wherein said biofilm is of the genus Staphylococcus, Streptococcus, Enterococcus, Mycobacterium. Listeria, Pseudomonas, Serratia, Escherichia, Klebsiella, Haemophilus, Enterobacter, Proteus, Acinetobacter, Neisseria, Stenotrophomonas, Citrobacter, Salmonella, Morganella, Corynebacterium, Pasteurella, Stenotrohonomas, Aeromonas, Bordatella, Providencia, Bacteroides, Shigella, Legionella, Vibrio, Yersinia, Helicobacter, Propionibacterium, Gardnerella or Campylobacter.

73. The composition of claim 61, wherein said infection is resistant to antibacterial agent agents.

74. The composition of claim 73, wherein said infection is a chronic infection or persistent infection.

75. The composition of claim 74, wherein said infection is endocarditis, osteomyelitis, an infection in a neutropenic subject or a biomaterial infection.

76. The composition of claim 61, wherein said antibacterial agent is selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants.

77. The composition of claim 76, further comprising a first and a second antibacterial agent selected from the group consisting of macrolides, ketolides, tetracyclines, chloramphenicols, lincosamides, oxazolidinones, rifamycins, aminoglycosides, glycopeptides, daptomycins, fusidic acids, sulphonamides, cycloserines, β-lactams, diaminopyrimidines, isonicotinic acids, nitrofurans, antiseptics and disinfectants; wherein said first and said second antibacterial agents are chemically distinct compounds.

78. A method for increasing the bactericidal action of an antibacterial agent comprising:
(a) contacting a bacterial cell with an antibacterial agent; and
(b) contacting said bacterial cell with an acyl hydrazide potentiator, an oxy amide potentiator, or an 8-hydroxy quinoline potentiator,
wherein said potentiator promotes the intracellular accumulation of a metal.

79. The method of claim 78, wherein said metal is iron, copper or manganese.
